(19) United States
(12) Patent Application Publication
(10) Pub. No.: US 2026/0000154 A1
(43) Pub. Date: Jan. 1, 2026

(54) DIABETIC FOOT HEALING SYSTEM AND METHOD

(71) Applicant: DEAN VAYSER, San Diego, CA (US)

(72) Inventor: DEAN VAYSER, San Diego, CA (US)

(21) Appl. No.: 19/253,685

(22) Filed: Jun. 27, 2025

Related U.S. Application Data
(60) Provisional application No. 63/664,876, filed on Jun. 27, 2024.
(60) Provisional application No. 63/791,202, filed on Apr. 18, 2025.
(60) Provisional application No. 63/801,747, filed on May 7, 2025.

(51) Int. Cl. A43B 7/14; A61B 5/103; A61F 5/30

(52) U.S. Cl. CPC A43B 7/14; A61F 5/30; A61B 5/1036

(57) ABSTRACT

The present invention is a diabetic healing system for the treatment of diabetic foot ulcers. This device promotes the healing process of diabetic foot ulcers by completely offloading the pressures that an ulcer may be subjected to regardless of its size, shape or location on the foot/ankle and/or toes. The device is made of a custom rigid outer shell and a custom inner lattice structure that fully supports, stabilizes and conforms to all aspects of a patient's condition, anomalies and disfigurement. It completely offloads (zero contact) any ulceration regardless of the location on the foot, ankle or toes. The offloading occurs through the use of recessed areas that can be incorporated directly into the lattice structure, or can be included in a “plug” that fits within a small aperture in the lattice structure.

### RELATED APPLICATIONS

This application claims priority to U.S. Provisional Application No. 63/801,747, for “Diabetic Healing Shoe and Method” filed on May 7, 2025, and currently co-pending; and claims priority to U.S. Provisional Application No. 63/791,202, for “Diabetic Healing Shoe and Method” filed on Apr. 18, 2025, and currently co-pending; and claims priority to U.S. Provisional Application No. 63/664,876, for “Diabetic Healing Shoe and Method” filed on Jun. 27, 2024, and currently co-pending. All of which are fully incorporated herein by reference.

### FIELD OF THE INVENTION

The present invention relates to a custom-made shoe that facilitates the treatment and prevention of diabetic foot ulcers, regardless of the location on the foot, and the method of manufacturing.

### BACKGROUND OF THE INVENTION

People with diabetes often develop peripheral neuropathy, which impairs their ability to sense pain and pressure. As a result, they may not feel injuries—such as stepping on a sharp object—leading to skin punctures and the formation of diabetic foot ulcers (“DFUs”), a condition affecting nearly 300 million people worldwide. Neuropathy also contributes to foot deformities and dry skin, increasing the risk of callus formation. Repetitive stress from walking or minor injuries can cause these calluses to break down and develop into DFUs. Additionally, many individuals with diabetes have peripheral artery disease (PAD), which reduces blood flow and further impairs healing. Regardless of the cause, DFUs are highly prone to developing infections, prolonged hospitalization and can ultimately lead to foot amputation.

DFUs are a significant global health issue, contributing to high morbidity, mortality, and economic burden. Globally, approximately 6.3% of individuals with diabetes develop DFUs, with higher rates observed in North America (13%) followed by Africa (7.2%), Asia (5.5%), Europe (5.1%), and Oceania (3.0%). Each year, between 18.6 and 26.1 million new DFU cases are reported, and 19% to 34% of people with diabetes are expected to develop a foot ulcer during their lifetime. In the United States, about 1.6 million individuals develop DFUs annually, and 8% of diabetic Medicare beneficiaries are affected. These ulcers are often complicated by infection, with around 50% becoming infected and 20% of these infections leading to partial or full foot amputations. DFUs also have a high recurrence rate, with up to 58% recurring within three years and up to 65% within five years.

The economic burden of DFUs is substantial. In the United States, DFUs add an estimated $9 to $13 billion annually to healthcare costs, with patients with DFUs incurring approximately $58,000 in annual healthcare costs compared to $17,000 for diabetic patients without foot ulcers. Treatment costs for DFUs average around $24,226 per episode, with minor amputations costing about $10,468 per patient-year and major amputations reaching up to $100,000 per procedure. On a global scale, the direct costs of DFU treatment are projected to reach $74 billion annually, with costs varying by region, ranging from $102 in Tanzania to $3,959 in the United States for less severe cases. In Europe, the average cost per DFU patient is €4,888, with hospitalization accounting for 88% of expenses.

Key risk factors for DFUs include peripheral neuropathy, peripheral arterial disease (PAD), foot and digital deformities, and trauma from ill-fitting shoes that cause repetitive stress. Peripheral neuropathy affects up to 80% of individuals with DFUs, leading to a loss of sensation in the feet and making injuries go unnoticed. PAD, present in approximately 50% of DFU cases, impairs blood flow to the lower extremities, hindering wound healing and increasing the risk of infection. Foot deformities, such as bunions or Charcot foot, can create pressure points that are prone to breakdown, while trauma from minor injuries or repetitive stress can lead to ulcer formation, especially when healing mechanisms are impaired due to diabetes. Neuropathy also contributes to foot deformities and dry skin, increasing the risk of callus formation. Repetitive stress from walking or minor injuries can cause these calluses to break down and develop into DFUs.

Regardless of the cause, DFUs that are not timely treated, are highly prone to developing infections, prolonged hospitalization and can ultimately lead to foot amputation.

DFUs are associated with alarmingly high mortality rates, with 30% to 50% of affected individuals dying within five years of diagnosis and up to 70% within ten years. These figures are comparable to, or worse than, mortality rates for several common cancers, including colorectal, breast, and prostate cancer. Mortality risks are even higher following amputations: 5-year mortality reaches 60% to 74% after a major (e.g., above-ankle) amputation, while even minor amputations are linked to 30% to 50% mortality over five years. Key factors contributing to higher mortality include advanced age, poor glycemic control, chronic kidney disease, cardiovascular disease, presence of infection or gangrene, and lower socioeconomic status. Clinically, DFUs should be viewed not only as limb-threatening but also as life-threatening conditions that signal systemic vascular disease and warrant aggressive multidisciplinary management.

Preventing and effectively managing diabetic foot ulcers (DFUs) is critical to reducing their substantial health and economic burdens. DFUs are a leading cause of lower-limb amputations and are associated with high morbidity, mortality, and healthcare costs. Early detection through regular foot examinations, along with proper glycemic control, plays a foundational role in prevention. Equally important is patient education, which empowers individuals to recognize early warning signs, adopt protective behaviors, and adhere to recommended treatments. A proactive, multidisciplinary approach—combining medical oversight, technological innovation, and patient engagement—is essential for minimizing DFU incidence and improving long-term outcomes.

For decades, the treatment and management of diabetic foot ulcers (DFUs) has largely centered on the development of advanced topical and biological wound care products, such as cellular or skin substitutes and stem cells. They often come at significant high cost yet offer only slightly above average efficacy. While comparatively less effort has been directed toward improving offloading techniques—a critical component in ulcer healing.

These advanced wound products are typically applied directly to the wound bed, with the goal of stimulating cell migration and promoting tissue repair. They vary widely in composition and mechanism of action: some contain living cells that support the healing process, while others are acellular; some are cryopreserved, others dehydrated; some include antimicrobial agents, while others are infused with collagen or anti-inflammatory compounds.

In addition to these products, secondary dressings—such as alginates, collagens and foams—are often used in conjunction to support the wound environment and promote healing. Today, the wound care market is saturated with an estimated thousands of different wound dressings and numerous costly advanced cellular products.

Despite this extensive variety, clinical evidence does not consistently demonstrate the cost-effectiveness of many of these options. In many cases, the efficacy of these dressings remains comparable to more basic treatments, raising concerns about their value in routine clinical practice. On average, clinical trials data suggest that the healing efficacy of most advanced products for DFUs hovers around 64%, regardless of the specific formulation used.

In contrast, the area of offloading—the practice of relieving pressure from the wound site—has seen minimal innovation over the past 30 years, despite its well-established importance in promoting DFU healing.

There are numerous factors that can impact the wound healing process of a diabetic foot ulcer. The primary factor that impedes or delays the healing of a diabetic foot wound—regardless of its location—is the presence of persistent, elevated pressure on the wound bed and the surrounding skin. This sustained mechanical stress disrupts tissue repair, prolongs inflammation, and significantly increases the risk of wound progression and complications. Additionally, inadequate blood flow, poor nutritional status, lack of compliance with medical recommendations, foot deformity, and age also contribute to delayed healing. For years, the biggest challenge in the treatment and healing of diabetic foot ulcers has been to find a solution that can properly eliminate all (plantar, dorsal, posterior, medial and lateral) pressures effecting the entire foot, including the toes.

Constant and repetitive forces on the foot are damaging the normal cellular migration and creating a chronic state of inflammation at the wound site. This causes senescence in cell migration, skin growth, thus delaying wound healing. The delay in wound healing puts patients at a very high risk of potentially getting an infection which could ultimately lead to an amputation of the extremity.

Offloading—the reduction or elimination of mechanical pressure at the site of a diabetic foot ulcer—is arguably the single most important factor in successful wound healing. Without adequate offloading, even the most advanced topical treatments are unlikely to achieve optimal outcomes, as continued pressure exacerbates tissue breakdown, impairs perfusion, and hinders the healing process.

The current “gold” standard for offloading is the use of total contact casts (“TCCs”), which are widely recognized as the most effective method. However, their adoption in clinical practice remains low due to practical barriers such as patient discomfort, mobility limitations, provider time constraints, and the need for specialized training. As a result, less effective alternatives like removable cast walkers (“RCWs”) or therapeutic footwear are often used, though they rely heavily on patient adherence—which is frequently suboptimal. These devices are generic, cumbersome, and have a very high rate of patient non-compliance. Their method of offloading is to “redistribute” the pressure but not fully offload.

Over the past three decades, there has been a notable lack of innovation in offloading strategies. While wound care products have proliferated, advancements in biomechanically engineered offloading devices, smart offloading technologies (e.g., pressure sensors, adherence monitoring), or personalized gait-modifying interventions have lagged. This gap presents a significant opportunity: rebalancing research and development priorities toward evidence-based offloading solutions could dramatically improve healing outcomes and reduce the burden of DFUs on patients and healthcare systems.

To meaningfully advance DFU care, future efforts must prioritize integrated treatment strategies where effective offloading is not an afterthought, but a foundational component of the care plan.

Historically, the TCC has been regarded as the “gold standard” for offloading diabetic foot ulcers, based on research conducted over 30 years ago. The underlying principle was biomechanically sound: by increasing the contact area of the plantar surface of the foot, the same body weight (Force) would be distributed over a greater surface area (Area), thus reducing pressure on the wound site (P=F/A).

However, despite this theoretical strength, the practical application of the TCC has significant limitations. The casting process is time-consuming, labor-intensive, and requires specialized staff with training in both the technique and the materials involved. It is messy and technically demanding, making it inaccessible to many general medical practitioners who lack the necessary support infrastructure. Furthermore, the TCC primarily redistributes pressure on the plantar surface of the foot but fails to address pressure concerns on other parts of the diabetic foot or ankle that are also vulnerable to ulceration.

To meaningfully advance DFU care, future efforts must prioritize integrated treatment strategies where effective offloading is not an afterthought, but a foundational component of the care plan.

Crucially, the TCC is not designed for preventive care. Its restrictive nature and intensive application process make it unsuitable for long-term or proactive use in patients at high risk for ulcer development. These limitations underscore the urgent need for more practical, scalable, and comprehensive offloading solutions in both treatment and prevention.

In addition to the logistical and clinical limitations of total contact casts (TCCs), they present significant challenges for elderly patients or those with gait instability. These individuals are already at a heightened risk for falls and hip fractures, making the use of rigid, cumbersome casts a potentially hazardous choice. Even when applied correctly, TCCs interfere with essential daily activities such as walking, bathing, and driving, further reducing their practicality and patient adherence.

As a result of these limitations, many clinicians default to the use of prefabricated, generic offloading boots. However, these solutions are far from ideal. The prevailing approach—fitting the foot into a standardized boot or brace—fails to account for the natural anatomical variability of patients. Diabetic patients often experience foot deformities, swelling, or asymmetries that make it difficult to achieve proper fit using standardized sizing (e.g., small, medium, large). A person with a size 10 foot may still not fit comfortably into a size 10 or medium boot, especially if edema or structural abnormalities are present.

Even when fitted appropriately, these boots are typically heavy, rigid, and awkward to walk in. For patients with existing mobility impairments or poor balance, such design features can worsen the risk of falls and reduce willingness to wear the devices consistently. Poor fit, discomfort, and interference with daily life all contribute to a high rate of patient non-compliance—ultimately undermining the therapeutic goals of offloading.

A recent study that involved 5,000 patients identified key factors that contribute to low adherence with prescribed diabetic footwear. These key factors include: (1) difficulty with donning and doffing—many users found the footwear challenging to put on or remove, particularly the elderly and those with limited mobility or dexterity; (2) stigmatization—the medical appearance of current devices made users feel visibly marked as having a health condition, leading to social discomfort or embarrassment; (3) lifestyle disruption—patients expressed that the devices hindered their ability to perform normal daily activities such as working, driving, or walking comfortably; and (4) lack of symptoms—the absence of foot pain led many to underestimate the severity of their condition and the importance of wearing preventive footwear. This study highlights the urgent need for diabetic footwear solutions that are easy to use, discreet, and compatible with daily life, while also addressing the psychological and behavioral dimensions of patient adherence.

These issues highlight the pressing need for more individualized, ergonomic, and user-centered offloading solutions. The importance of addressing DFUs cannot be overstated: they significantly impair quality of life, increase the risk of hospitalization and amputation, and impose a substantial burden on healthcare systems. Comprehensive foot care programs, paired with innovative offloading strategies, are critical to reducing the incidence, recurrence, and economic costs of diabetic foot ulcers—while also improving long-term patient outcomes.

In light of the limitations and clinical gaps outlined above, there is a clear and urgent need for a paradigm shift in diabetic foot ulcer (DFU) management. A revolutionary diabetic foot healing system should be designed to deliver both effective treatment and proactive prevention by focusing on true personalization. Rather than forcing a patient's foot into a generic, prefabricated device, the system must be capable of creating a fully customized offloading solution tailored to the unique shape, size, and deformities of each individual's foot and ankle. The device must also be able to support full integration into daily life of the patient to ensure a high rate of compliance. This approach would fundamentally reverse the current paradigm: instead of fitting the foot to a device, we must design a system that fits the device to the foot.

To be truly transformative, this system should be intuitive and accessible to all patients—regardless of age, mobility, or technical ability. Whether a patient is 18 or 85 years old, the device should be easy to don and doff, functionally seamless, and aesthetically designed to resemble and feel like ordinary footwear. Stability, comfort, and cosmetic appeal are essential to driving adherence and improving quality of life.

Moreover, the device must support full integration into daily life. It should be wearable in bed, in the shower, around the house, outside, and even while driving—eliminating the need for constant removal and reapplication, which often disrupts compliance. The system should enable continuous offloading without impeding the patient's independence or routine activities.

Such a solution—personalized, practical, and lifestyle-compatible—would represent a breakthrough in the treatment and prevention of DFUs, addressing the current limitations in offloading technologies and significantly advancing both clinical outcomes and patient satisfaction.

### SUMMARY OF THE INVENTION

Generally, the present invention addresses the limitations and clinical gaps as outlined and provides a comprehensive diabetic foot healing system that addresses both the treatment and prevention of DFUs, regardless of their location, through an integrated, patient-specific approach—all delivered within a single, multifunctional device. There are two different core components for the system in this embodiment. The first is the healing component, which is the portion of the device that provides complete and total offloading through a non-contact interface that fully suspends the ulcerated area from any contact other than dressings. By eliminating all pressure at the wound site, it facilitates optimal healing conditions. The healing portion of the system is dynamically fabricated and refitted at regular intervals, based on the wound's healing trajectory (size, depth, and rate of closure), ensuring continuous customization throughout the healing process. The second is the prevention component, which is a permanent, long-term device that is fabricated to prevent ulcer recurrence once healing of the DFU is achieved. This preventative solution redistributes and decreases high pressure points on the foot (regardless of its location). In addition, it corrects gait instability and provides total contact to the non-ulcerated foot surface, thus preventing callus formation which could lead to ulcer recurrence. Its design maintains normal biomechanical function, enhances stability, and protects high-risk areas—minimizing the likelihood of future ulcer formation.

The present invention includes a custom diagnostic platform that collects detailed, high-resolution scans and biometric data of the patient's foot and ankle. This includes, but is not limited to, three-dimensional structure scans to assess the patient's anatomy and any deformities; plantar pressure mapping to identify high-load zones; and gait analysis to evaluate limb discrepancies, load imbalances, and stability challenges. The platform superimposes this data into a hybrid digital foot model, generating a precise map that overlays real-time pressure points onto the anatomical image of the patient's foot—including ulcer shape, size, and location. The platform superimposes this data into a hybrid digital foot model, generating a precise map that overlays real-time pressure points onto the anatomical image of the patient's foot—including ulcer shape, size, and location.

This information is then used to design and fabricate a fully customized diabetic foot device that conforms to the foot and ankle like a glove, addressing each patient's unique anatomy and offloading needs. The ulcer region is suspended in a zero-contact zone, while the rest of the foot, the non-ulcerative region, is completely wrapped in a functional equivalent of a total contact cast, but with significantly enhanced benefits that includes, but is not limited to: (1) true customization for deformities and limb length discrepancies; (2) no mess, no casting materials, and no specialized application techniques; (3) full mobility for the patient to ensure that they can walk, drive, shower, and sleep while wearing the device; and (4) an aesthetic design for daily wear, promoting dignity and normalcy. The invention thus revolutionizes DFU management by offering a complete, user-friendly, data-driven solution that not only expedites healing but also actively prevents recurrence—empowering patients to reclaim mobility, independence, and quality of life.

The Diabetic Foot Healing System of the present invention offers a significant advancement in both the healing and prevention of diabetic foot ulcers through a data-driven, individualized approach. The therapeutic process begins with a comprehensive assessment of the patient's lower limb using advanced diagnostic technologies. The initial phase involves high-resolution 3D limb scanning combined with a thorough gait analysis, conducted under both static and dynamic conditions. This dual-stage analysis measures: (1) plantar pressure distribution in motion and at rest; (2) gait abnormalities and instability; (3) limb-length discrepancies; and (4) full 3D anatomical mapping of the foot and ankle to identify and address ulcers that are not found on the plantar aspect of the foot.

The scanning process generates an ultra-detailed digital model of the patient's foot and ankle, capturing every contour, deformity, swelling, or musculoskeletal abnormality specific to the individual. This 3D anatomical image is superimposed with plantar pressure data to create a hybrid visual map.

Proprietary software then analyzes the combined data to identify critical corrections needed for achieving gait stability, compensating for or supporting structural deformities, and most importantly, offloading any diabetic ulcer with precise, zero-contact mapping.

The result is a fully custom-fabricated device that achieves total contact with the non-ulcerated portions of the foot and ankle—including the toes—ensuring optimal weight distribution, balance control, and targeted protection. The ulcer site itself is fully suspended with no device contact, tailored exactly to its shape and size.

Further enhancing its therapeutic potential, the system accommodates integration with: (1) small secondary dressings, advanced biologics (skin substitutes); (2) negative pressure wound therapy devices (NPWT) where appropriate; (3) embedded sensors to monitor pressure, temperature, wear time (compliance), and potentially early warning signs of recurrence. Every millimeter of the foot is accounted for during the design phase. Whether the patient presents with long toes, short toes, a wide or narrow foot, high arch, flat foot, or complex deformities, the resulting device is precision-engineered to conform completely to the foot's anatomy while delivering comprehensive offloading of the ulcer and proactive correction of biomechanical deficiencies.

In a preferred embodiment, the custom therapeutic device comprises two main components: an outer rigid shell, one for the dorsal part of the foot and the other for the plantar part, and inner custom lattices, one for the dorsal part of the foot and the other for the plantar part, that fits securely within the inside of the outer shell. The foot and ankle will be nestled inside this device with both parts of the outer shell securely protecting the entire foot like a “clam”. The dorsal part of the outer shell will be hinged on one side to the plantar outer shell and securely fastened to the other side of the plantar outer shell creating a snug and custom fit covering every aspect of the foot/ankle, including the toes. The foot will sit firmly and securely inside the device with zero movement, except at the ankle joint.

The Outer rigid shell is designed to protect and provide load bearing to the patients foot/ankle. The plantar portion will have a rocker shape to allow for easier forward propulsion and lessening the stresses at the mid portion of the foot. An optional rocker-bottom, non-slip plate can be clipped onto the plantar surface for safe outdoor ambulation. This plate is removable, enabling patients to adapt the device for indoor vs. outdoor use.

The Inner custom lattice will be nestled within the outer rigid shell. It will conform precisely to the entire foot's surface, delivering stability and correction at all points except the wound site, which is fully suspended in a zero-contact zone. A custom “plug”, fabricated to the exact shape and size of the ulceration will sit inside the inter lattice ulcer opening. This method will allow for dressings, biologics, negative pressure foams, and other products to be easily placed inside the “plug” and changed out as needed. The “plug” can be removed and/or replaced as needed, based on changes to the ulcer size/shape or need for particular dressings. The interchangeable “plug” component can be easily fabricated at physician's office leaving both the outer and inner components in place for the duration of the treatment process. The modular treatment process allows for ease in customization and maintenance.

Throughout the course of treatment, the patient's foot will undergo regular clinical evaluations at designated intervals. Each examination will assess the healing progress of the diabetic foot ulcer, and findings will be thoroughly documented. The “plug” can be refabricated approximately every two weeks, or as clinically indicated, to reflect changes in the wound size and healing status. This modular design allows the offloading architecture to adapt in real time to the evolving needs of the patient. At each stage of healing the ulcer area decreases, expanding the available plantar surface for safe weight-bearing. Consequently, the offloading cavity within the device is resized to maintain zero contact with the healing tissue. The pressure redistribution model is then recalibrated, increasing overall plantar surface area engagement, which reduces focal stress concentrations and accelerates recovery. These repetitive scans continue until the DFU is fully healed.

This gradual reintegration of weight-bearing zones not only enhances wound healing through biomechanical optimization but also improves gait symmetry and function over time. The ultimate therapeutic endpoint is complete ulcer resolution, at which point the patient transitions into the preventative phase of care. In this phase, a new long-term custom device may be fabricated using the same diagnostic data, adjusted for the fully healed foot, to prevent recurrence and maintain optimal pressure distribution during daily activities.

The preventative phase of the Diabetic foot treatment system will be initiated once the DFU is fully healed. In the preventative phase, the patient undergoes a final pressure foot scan to evaluate pressure distribution across the entire plantar surface and identify any notable changes. Based on the scan data—along with previously assessed factors such as gait abnormalities or limb length discrepancies—a custom inner shell is fabricated. This shell may be inserted back into the patient's existing outer shell that was previously used during the healing phase. In addition, data taken from the pressure scan can be used to fabricate a custom “insole”, that patient can use in their own diabetic footwear. The custom inner shell or a custom insert is tailored to the patient's unique anatomical features and is designed for long-term use and prevention. Its primary function is to redistribute pressure evenly, thereby eliminating localized high-pressure zones—the most common cause of diabetic foot ulceration.

In an improved embodiment of the diabetic foot treatment system, the inner lattice is equipped with a liner array of sensors that will detect pressure spikes or anomalies, sense changes in temperature, and other related data which could indicate a wound recurrence, or conditions that may lead to a recurrence. Using telemetry technologies known in the art, the diabetic foot treatment system of the present invention can notify the treating physician of potential new problems, or can be downloaded during a routine visit to the doctor's office.

In order to facilitate the daily wearing of the diabetic foot treatment system of the present invention, each outer shell can accommodate a separate generic rocker bottom plate which can be snapped onto the plantar plate. A patient wearing a plantar plate can easily “clip into” the bottom plate when they wish to go outside or drive. This lower surface of the plantar plate is coated with a protective rubber base to provide an increased level of friction and guard against slips and falls.

In a preferred embodiment, attaching the bottom plate to the plantar plate is similar to putting your ski boot onto the ski bindings, such as inserting the toe end of the plantar plate into the front of the bottom plate, and then pushing the heel end of the plantar plate onto the back of the bottom plate until it snaps together. When returning home, the patient may remove the bottom plate from the plantar plate simply by using one foot to push down on the back end of the bottom plate on the other foot to unclip it from the plantar plate. The diabetic foot treatment system of the present invention allows patients to always have protection inside or outside the house, improving compliance with offloading, greatly reducing the duration of foot ulcer treatment, and greatly improving the outcomes of diabetic foot ulcer treatment.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a system level diagram showing the diabetic foot treatment system of the present invention and includes a foot pressure platform and three-dimensional foot scanner connected to a server that has access to cloud resources including remote manufacturing information, is connected to a local design workstation, and analyzes the data from the foot pressure platform and three-dimensional foot scanner to output details and data to a three-dimensional print station designed to print all components of the device for use in the present invention to treat diabetic foot ulcers;

FIG. 2 is a side-by-side image of a patient's foot having a diabetic foot ulcer on the ball of the foot, and the corresponding design image providing a contour map for the pressure zones on the patient's foot as created by the data taken from the pressure plate as designed;

FIG. 3 is a side view of a patient's foot being placed into a custom manufactured inner lattice shell designed to minimize pressure on an affected ulcer area and also showing the attachments anchors to receive rubber or silicone sheets to facilitate attachment of the plantar plate to the patient's foot;

FIG. 4 is a side view of a patient's foot that has been placed in the custom manufactured plantar plate and showing a complete contact surface between the foot and the plantar plate except for the area that is designated a low or no pressure zone adjacent and corresponding to the diabetic foot ulcer location;

FIG. 5 is a side view showing a patient's foot that has been placed in the custom manufactured plantar plate and secured in place using rubber or silicone sheets attached to the anchors around the periphery of the plantar plate to secure the plantar plate to the patient's foot yet provide for free movement of the foot relative to the lower leg and ankle;

FIG. 6 is a side view showing a patient's foot that has been placed in the custom manufactured plantar plate and secured in place using rubber or silicone sheets and also equipped with a walking cover that is designed to provide a clean and removable higher friction surface to facilitate a patient's walking outside and driving and living a normal life;

FIG. 7 is a flow chart showing an exemplary method of analysis, design and manufacturing of the custom manufactured plantar plate for the patient's treatment of a diabetic foot ulcer;

FIG. 8 is a system level diagram of an alternative embodiment of the present invention being manufactured;

FIG. 9 is a top view of an alternative embodiment of the present invention with a custom fitted dorsal plate component attached to the plantar outer shell;

FIG. 10 is a side view of an alternative embodiment of the present invention showing how the custom dorsal plate is attached to the plantar plate;

FIG. 11 is a side view of an alternative embodiment of the present invention showing the walking cover on the plantar plate;

FIGS. 12A through 12D are top views of the plantar plate portion of the present invention that shows how the pressure zones get smaller as the diabetic ulcer heals over time;

FIG. 13 shows a top and bottom view of an alternative embodiment of the plantar plate that has an aperture for a physician to provide secondary treatment methods directly to the diabetic ulcer;

FIG. 14 is a top view of an alternative embodiment of the plantar plate that a multitude of different sensors built into it;

FIG. 15 is a flow chart showing an exemplary method of analysis, design, and manufacturing of the custom plantar plate and the custom dorsal plate;

FIGS. 16A through 16D show section views of the custom plantar plate at different stages of the healing process for the diabetic healing shoe;

FIG. 17 is a section view of the custom plantar plate with medicants in the recessed areas;

FIG. 18 is a section view of the custom plantar plate that was designed to accommodate a negative pressure wound therapy device;

FIG. 19 is a close-up section view of the custom plantar plate support blocks in the recessed area;

FIG. 20 is a section view of the custom plantar plate having shock absorbers installed in the recesses;

FIGS. 21A and 21B are side views of a fully assembled alternative embodiment of the diabetic healing shoe;

FIG. 22 is a side view of the inner lattice structure of an alternative embodiment of the diabetic healing shoe;

FIG. 23 is a system level diagram of an alternative embodiment of the present invention being manufactured;

FIG. 24 is a top view of the inner lattice structure of an alternative embodiment of the diabetic healing shoe;

FIGS. 25A through 25C are section views of the bottom of the of the inner lattice that shows the different stages of the healing process for an alternative embodiment of the diabetic healing shoe;

FIG. 26 is a side view of the inner lattice structure of an alternative embodiment of the diabetic healing shoe;

FIGS. 27A through 27C are section views of a side wall of the lattice shell that shows the different stages of the healing process for an alternative embodiment of the diabetic healing shoe where the diabetic ulcer is located on the foot in a location other than on the plantar surface;

FIG. 28 is a top view of the dorsum lattice of an alternative embodiment of the diabetic healing shoe;

FIGS. 29A through 29C are section views of the dorsum lattice that shows the different stages of the healing process for an alternative embodiment of the diabetic healing shoe;

FIG. 30 is a perspective view of the outer shell of an alternative embodiment of the diabetic healing shoe;

FIG. 31 is a perspective view of the lattice shell with a healing plug;

FIG. 32 is a side view of an alternative view of the outer shell of the diabetic healing shoe;

FIG. 33 is a side view of an alternative embodiment of the outer shell and lattice shell of the diabetic healing shoe; and

FIG. 34 is a flow chart that outlines an exemplary order of operations for the design and manufacturing of an alternative embodiment of the diabetic healing shoe.

### DETAILED DESCRIPTION

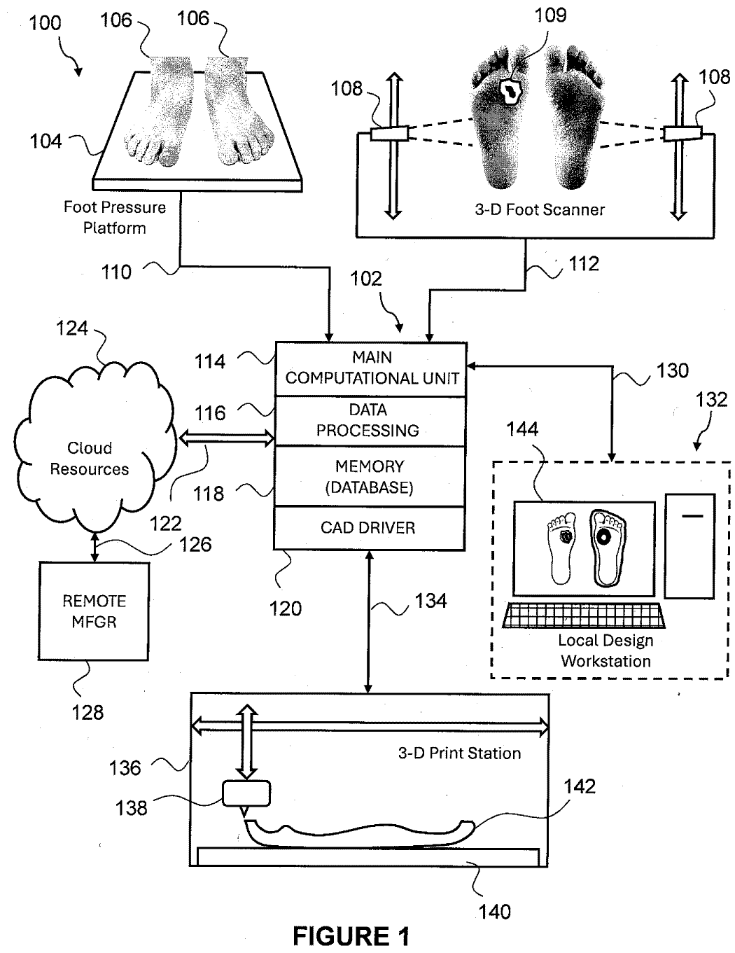

Referring initially to FIG. 1, the diabetic foot treatment system of the present invention is shown and generally designated 100. System 100 includes a main server 102, a foot pressure platform 104, and a 3D foot scanner 108. Foot pressure platform 104 is sized to have a patient stand on top of the pressure platform such that the foot pressure provides data to the system corresponding to the pressure points and the pressure profile of foot 106 on the ground. Foot pressure platform 104 can be anywhere from six to seven feet in length, but pressure platforms that are shorter or longer are fully contemplated herein. The length of foot pressure platform 104 ensures that the patient can walk back and forth along the platform so that the health care provider can collect all of the relevant data needed. The three-dimensional foot scanner includes one or more scanning heads 108 that optically map the entire surface of the foot/ankle in full, partial or non-weightbearing to identify the particular contour and natural shape, as well as any potential defects caused by diabetic foot ulcer 109.

The output from foot pressure platform 104 is connected with data line 110 and the output from the three-dimensional foot scanner on data line 112 is sent to server 102. Server 102 includes a main computational unit 114, a data processing unit 116 a memory or database 118 and a computer aided design driver 120. It is to be appreciated that a computer of this nature is generally known in the art so long as it can accomplish the tasks that are outlined herein and described in the method steps below.

Server 102 is connected via data link 122 to cloud resources 124. Cloud resources 124 can include a great deal of additional information regarding optimized treatment protocols, optimized pressure mapping techniques, as well as other information regarding the specific diabetic foot treatment system as it applies to specific patients. Additionally, a remote manufacturing facility 128 can also be attached to the cloud resources and can interface directly or indirectly with server 102 through data line 126.

Also connected to server 102 is local design workstation 132 via connection 130 to the server. The local design workstation 132 includes a computer system that allows a physician to actively input, design, modify, and finalize the particular 3-dimensional design for the diabetic foot treatment system of the present invention. The screen 144 of local design workstation 132 provides immediate visual verification of the design of plantar plate 142 so that the healthcare provider can check for accuracy. As will be described more completely below the output from the server 102 once approved is sent to a 3-dimensional print station 136 which forms a custom plantar plate 142 corresponding to the patient, and the patient's needs for offloading their weight in light of diabetic foot ulcers and the complications therewith. The three-dimensional print station 136 has print head 138 that can print along the X, Y and Z axis and is capable of printing polymers and foams on platform 140 of different composite material allowing the creation of layers in the plantar plate that vary in rigidity and durometer rating Server 102 sends the approved designs to print station 132 through data link 134.

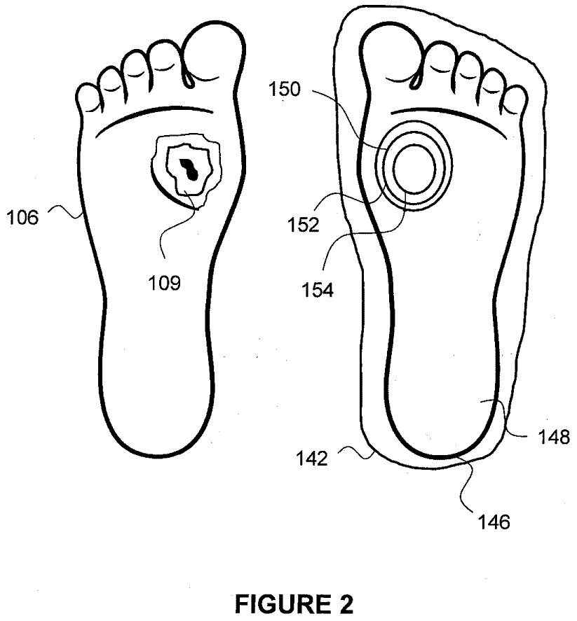

Referring now to FIG. 2 a representative image showing the bottom of a patient's foot 106 having a diabetic foot ulcer 109 next to the computer aided design of a plantar plate 142. From this image it can be seen that the mirror image of the foot as duplicated in the plantar plate has a perimeter region that is formed with a foot receiving area 146 that includes a foot contour area 148. The foot contour area 148 is designed to contact substantially all of the patient's foot surface area so that the majority of the weight of the patient is offloaded from the area that is affected by the ulcer. In this image there are areas that are related to the treatment of the diabetic foot ulcer and include, for example, a recessed area 150 that extends to the furthest perimeter of the ulcer 109, a slightly smaller recessed area 152 that corresponds to a more affected area of the diabetic foot ulcer, and a most recessed area 154 that corresponds to the most affected area of the diabetic foot ulcer.

When looking at the plantar plate 142, it can be seen that area 148 can be a full pressure zone, area 150 can be a lower pressure zone, area 152 can be a near zero pressure zone, and area 154 can be a no pressure zone. By utilizing this multilayer approach, the treating physician can customize the particular pressures that are applied to a wound area to facilitate healing as well as to provide sufficient space for the insertion of medication, treatment pads, or other conditions that will improve the healing process.

As can be appreciated from this view, custom plantar plate 142 can be designed to accommodate virtually any specific diabetic foot ulcer, or combination of ulcers, to provide an improvement to the healing areas and treatment areas for those patients. By having the ability to utilize a computer aided design system to customize the particular shapes and depths of pressure zones provides the treating physician with the ability to custom manufacture a plantar plate for virtually any diabetic foot ulcer.

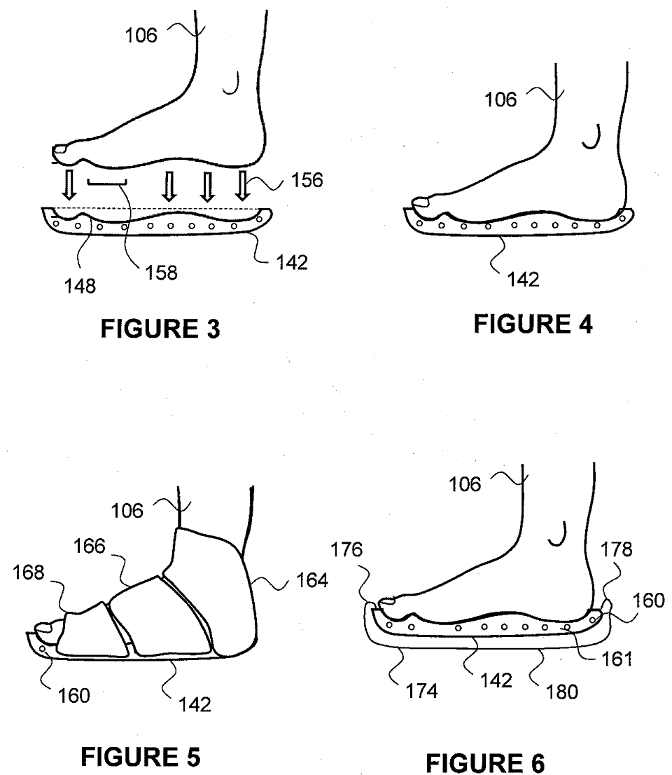

Referring now to FIG. 3, patient's foot 106 is shown as it is being inserted into the plantar plate 142. This is accomplished by moving in direction 156 such that the entire lower surface of the foot 106 contacts the upper contour surface 148 of plantar plate 142 there is, however, a number of pressure zones 158 on the foot which is corresponding to the placement of the pressure areas 150, 152, and 154 that have been identified in FIG. 2 above. To place foot 106, a patient just needs to lower foot 106 onto plantar plate 142 by following direction 156. FIG. 4 shows the patient's foot being securely received into the plantar plate 142 and indicates that there is no space between the foot and the plantar plate as seen in the side contour profile.

Referring now to FIG. 5, the plantar plate 142 is shown having anchors 160 around the perimeter. Also shown from this figure are a number of attachment sheets identified as 164, 166 and 168. From this view it can be seen that the plantar plate 142 is securely attached to the foot 106 of the patient. Attachment sheets can be manufactured from rubber, a polymer, or other material that is both secure and flexible so that a patient can walk freely with ample movement of the ankle to facilitate normal walking.

As shown the anchors secure the attachment sheets to the plantar plate 142 and it is to be appreciated that while they secure the attachment sheets in place, they may be removed relatively easily so that the treating physician can easily detach the plantar plate from their foot.

Referring to FIG. 6, a side view of the plantar plate 142 is shown to be equipped with a walking cover 174 that attaches to the plantar plate. Attachment of the walking cover 174 to the plantar plate 142 is accomplished by placing the toe end of the plantar plate into the receiver 176 of the walking cover 174 and then pushing the heel of plantar plate 142 into the heel end 178 of the walking cover 174. The walking cover 174 snaps on securely and provides a clean and removable higher friction surface 180 to facilitate a patient's walking outside and driving and living a normal life.

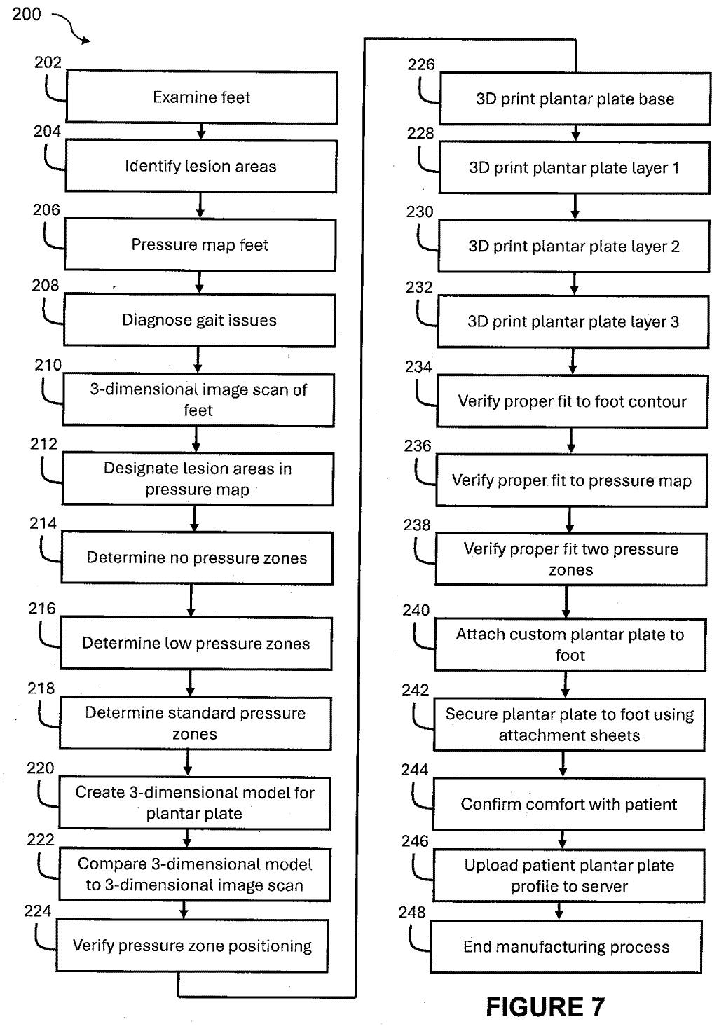

Referring now to FIG. 7, a flow chart shows the diabetic foot treatment system and its method of implementation and use, and is generally designated 200. The process begins at step 202 when a physician examines a patient's feet and determines that there is a diabetic foot ulcer that requires treatment. Oftentimes these diabetic foot ulcers drain fluid, may be bleeding, and often require that the physician debride the affected area. Once the lesion areas are identified and debrided in step 204, the patient's feet are then pressure mapped in step 206 to identify the natural pressure zones of the patient's feet in a normal standing, or in a normal gait, to provide the current pressure profile for the patient's feet including the pressures that are applied to the diabetic foot ulcer area.

In step 208 the physician can assess gait abnormalities which can also be addressed with corrections while manufacturing the custom plantar plate as described above. Once the gait issues have been detected in step 208, step 210 provides for the three-dimensional imaging scan of the feet. This provides an optical image of the feet without any pressure placed on the feet so as to get a natural contour of the foot for purposes of manufacturing the custom plantar plate. Once the three-dimensional image scan of the feet is completed, the physician will designate lesion areas in the pressure map in step 212.

As described above in relation to system 100 the local design workstation 132 is used to allow the physician to make manual adjustments and corrections of the pressure map in real-time for each particular patient. In addition to the identification of lesion areas, the physician also determines in step 214 the zero pressure zones for the patient's foot. These are areas that will have no physical contact from the plantar plate and in some cases may include a specific air gap between the surface of the plantar plate and the patient's lesion area. In addition to the zero pressure zones identified in step 214, the physician may also determine low pressure zones or standard pressure zones in steps 216 and 218. Once these zones have been identified in steps 214, 216, and 218, a three-dimensional model for the plantar plate is created in step 220. This three-dimensional model can be viewed on the local design workstation 132 and will provide the treating physician with an immediate visual verification 144 of the design of the custom plantar plate to ensure that these specific zero or low pressure zones have been accommodated for the benefit of the patients optimal healing protocol.

In step 222 the physician compares the three-dimensional model to the three-dimensional image scan using the local design workstation so that the physician can visually verify that the custom plantar plate appears to be satisfactory. During that analysis, the physician will verify the pressure zone positioning in step 224 and if accurate, the physician sends the three-dimensional design from the local design workstation through the server 102 to the three-dimensional printer 136 where the custom plantar plate 142 will be printed.

The three-dimensional printing of custom plantar plate 142 begins with the three-dimensional printing of the custom plantar plate base in step 226. On top of the base 140, the plantar plate layer one is printed in step 228, layer two is printed in step 230, and layer 3, if necessary, is printed in step 232. Once the custom plantar plate 142 is finished printing, the physician will properly fit the plantar plate to the patient's foot to verify proper fit and contour in step 234 and compliance with the requirements for no or low pressure zones 150, 152, and 154.

In addition to ensuring that the proper foot contour exists, the physician will also verify proper fit to the pressure map In step 236 to confirm that the no and low pressure zones 150, 152, and 154 are accurately positioned in the custom plantar plate 142 such that the treatment of the diabetic foot ulcer can begin. In the event that there are multiple lesions requiring multiple no or low pressure zones, those will be verified in step 238.

If the foot contour and pressure map are accurately reproduced in the plantar plate 142, in step 240 the custom plantar plate 142 is attached to the foot 106 and is then secured by using the attachment sheets 164, 166, and 168 at step 242 as described above. Because the attachment sheets can be attached with various degrees of tension, those may be adjusted for the comfort of the patient balanced with the need for a secure attachment of the custom plantar plate 142 to the foot. If the patient confirms an acceptable level of comfort in step 244 then the patient is dismissed with instructions to consistently wear the custom plantar plate 142 at all times unless instructed otherwise.

Following a successful imaging and scanning of a patient's foot, identification of various lesions, and manufacturing of a custom plantar plate, the patient digital plantar plate profile which includes the customer information along with the computer aided design information for the custom plantar plate is then uploaded to the server memory 118 for storage at step 246. Additionally, it can be sent from storage 118 to a cloud storage system 124, and perhaps to the remote manufacturer 128 for archival purposes, or for future manufacturing needs. Once the uploaded patient planter plate profile has been successfully saved, the manufacturing process for the custom plantar plate 142 is finished in step 248.

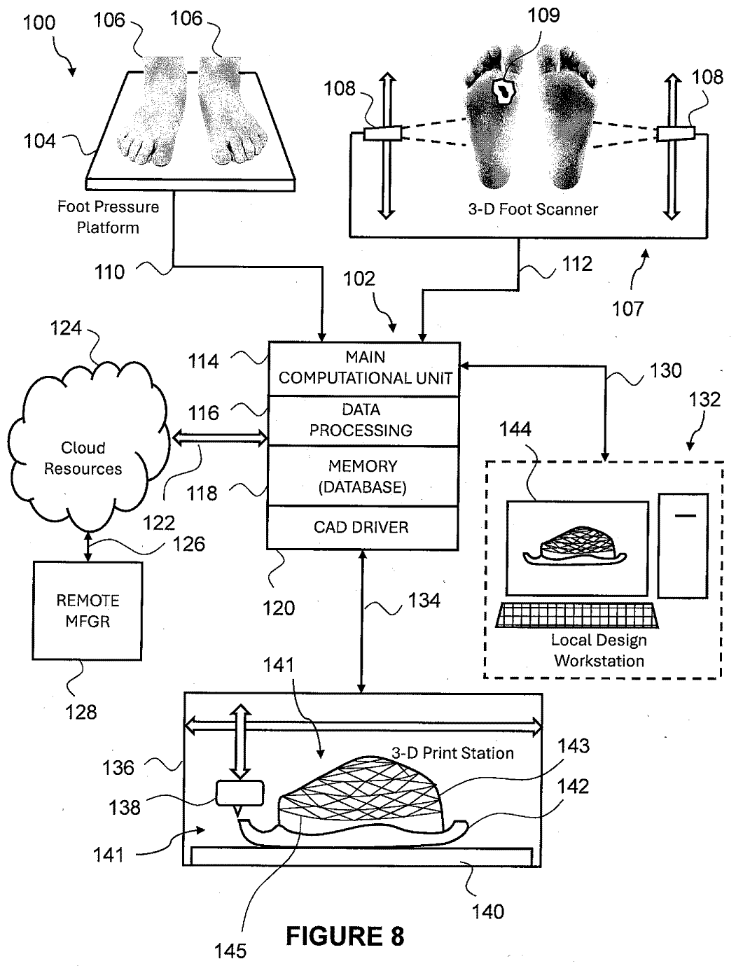

Referring now to FIG. 8, system 100 is shown again, but instead it is shown manufacturing an alternative embodiment of the present invention, diabetic healing shoe 141. Diabetic healing shoe 141 is made up of both dorsal plate 143 and plantar plate 142. Much of the system level discussion that was discussed in detail for FIG. 1 applies again here as system 100 is merely adapted to include the scanning, designing, and manufacturing of dorsal plate 143. The strength of dorsal plate 143 is directly attributed to lattice structure 145 which encases the dorsal portion of foot 106.

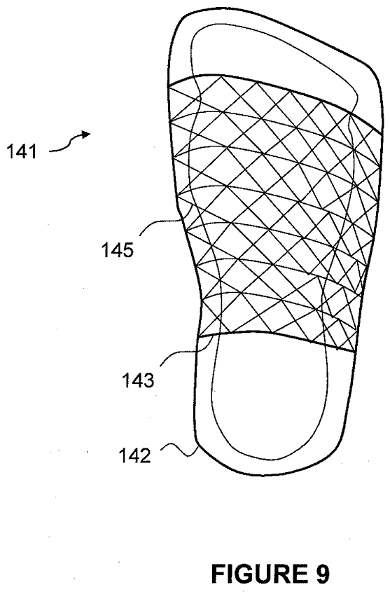

Referring now to FIG. 9, a top view of diabetic healing shoe 141 is shown. Of particular importance in this view is lattice structure 145 on dorsal plate 143. Lattice structure 145 provides the required strength and stability that is needed for dorsal plate 143. Additionally, lattice structure 145 secures foot 106 within plantar plate 142 and runs along the top of foot 106 and stops just shy of the ankle joint. This allows a patient to retain flexion of the ankle joint, allowing them to still use foot 106 in normal day to day activity. Lattice structure 145 is designed by the physician based on the results of the three dimensional scan of foot 106. This means that both dorsal plate 143 and plantar plate 142 are fully designed for each individual patient that uses diabetic healing shoe 141 and the shoe creates a snug fit onto foot 106, creating a fully customizable treatment plan.

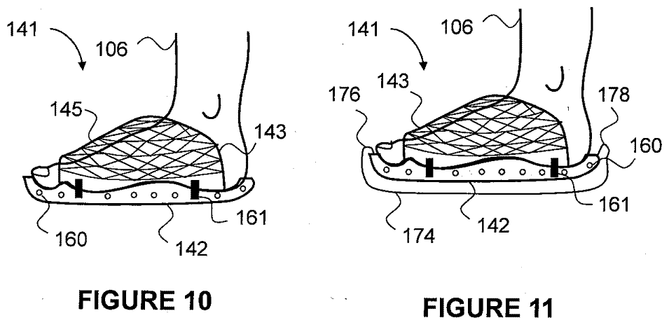

Referring now to FIGS. 10 and 11, a side view of diabetic healing shoe 141 is shown with and without walking cover 174. The high number of different anchors 160 provide a great of flexibility for where dorsal plate 143 can be attached to plantar plate 142. In both of these Figures, dorsal plate 143 is shown being attached to plantar plate 142 by hook and loop fasteners 161 at two different locations. However, it is fully envisioned that dorsal plate 143 can attached to plantar plate 142 by any fastening means known in the art that includes, but is not limited to, hooks and laces.

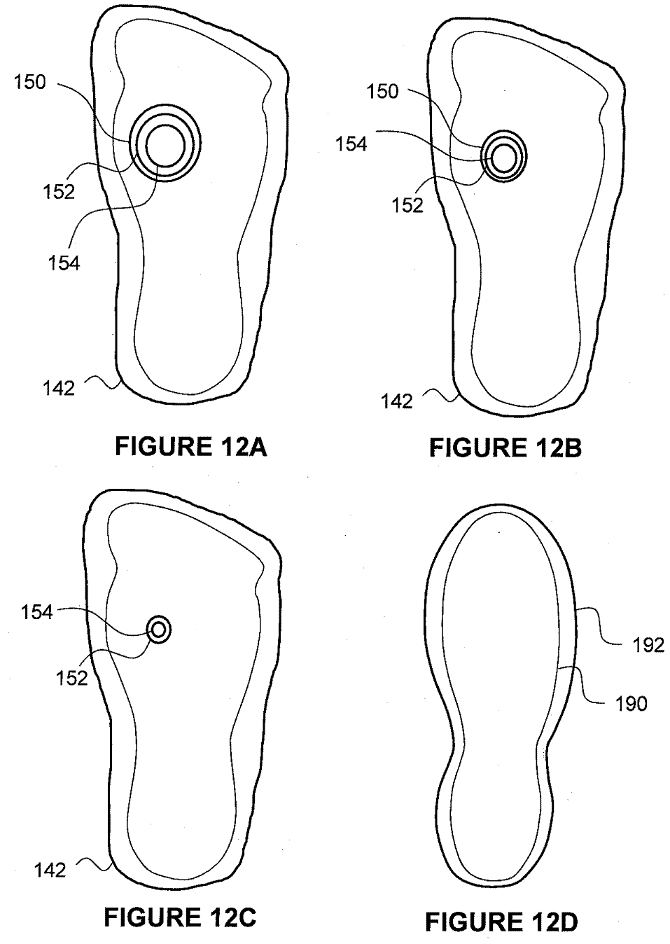

Referring now to FIGS. 12A-12D, multiple different plantar plates 142 are shown to highlight how plantar plate 142 may be customized during the healing process of a diabetic foot ulcer. Depending on the treatment plan, a physician may determine that it is necessary to have multiple different plantar plates 142 manufactured for the patient as the ulcer heals over time. Plantar plate 142 shown in FIG. 12A was manufactured for the initial diagnosis of a diabetic foot ulcer. In this view, the ulcer is at its most severe, and there are different offloading needs for the ulcer to start healing. This is reflected by the overall size of recessed area 150, slightly smaller recessed area 152, and most recessed area 154 being at their largest diameter to ensure that plantar pressure does not affect the diabetic foot ulcer. As the diabetic foot ulcer heals over time, then the requirements for offloading the plantar pressure changes. This is shown in FIGS. 12B and 12C by the subsequent reduction of size of the different recessed areas; there may be scenarios as shown specifically in FIG. 12C that recessed area 150 is no longer needed.

FIG. 12D shows diabetic insole 192 that can be used once treatment of the initial foot ulcer is successfully completed. Diabetic sole 192 is also able to offload plantar pressure and is designed to be a preventive treatment option for diabetic foot ulcers. The design process of diabetic insole 192 is the same as the design process for diabetic healing shoe 141. Diabetic insole 192 is able to fit within a patient's shoe just like any other custom orthotic sole. Foot contour area 190 operates functionally the same as foot contour area 148, and is designed to offload the specific plantar pressures of a patient based on the results of their foot scan and gait analysis.

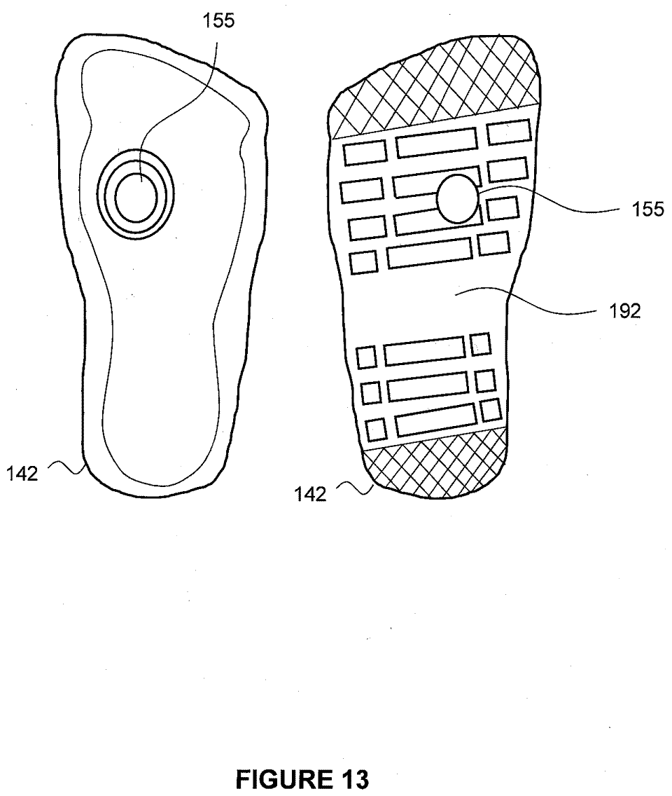

Referring now to FIG. 13, a top view and bottom view of plantar plate 142 is shown. Plantar plate 142 can also have a tread pattern 192 on the bottom surface to ensure that there is grip for a patient to rely on when walking in diabetic healing shoe 141. An alternative embodiment of most recessed area 154 is shown and instead of having a recessed area, aperture 155 is shown. Aperture 155 provides a physician with direct access to a diabetic foot ulcer. This allows multiple different secondary treatment options to be used in conjunction with diabetic healing shoe 141.

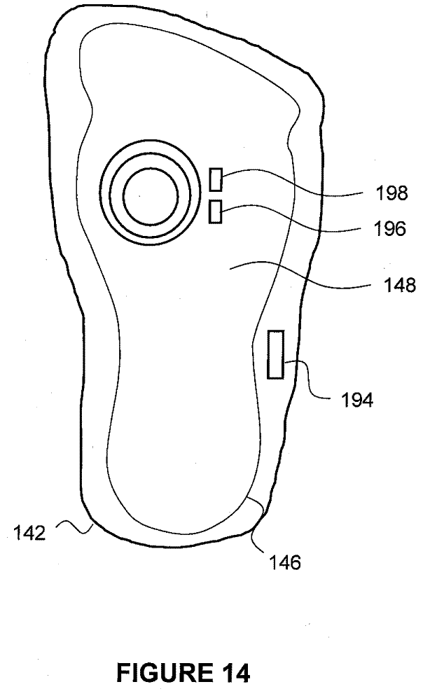

Referring now to FIG. 14, an alternative embodiment of plantar plate 142 is shown with multiple different sensors built in. Both pressure sensor 196 and temperature sensor 198 are built into foot contour area 148. Pressure sensor 196 and temperature sensor 198 monitor the temperature and pressure of the plantar plate 142 to make sure that ideal conditions are in place to facilitate the proper healing of a diabetic foot ulcer. Additionally, compliance sensor 194 is built into plantar plate 142 adjacent to foot receiving area 146. Having compliance sensor 194 built in allows a physician to monitor the patient to ensure that diabetic healing shoe 141 is being worn by the patient.

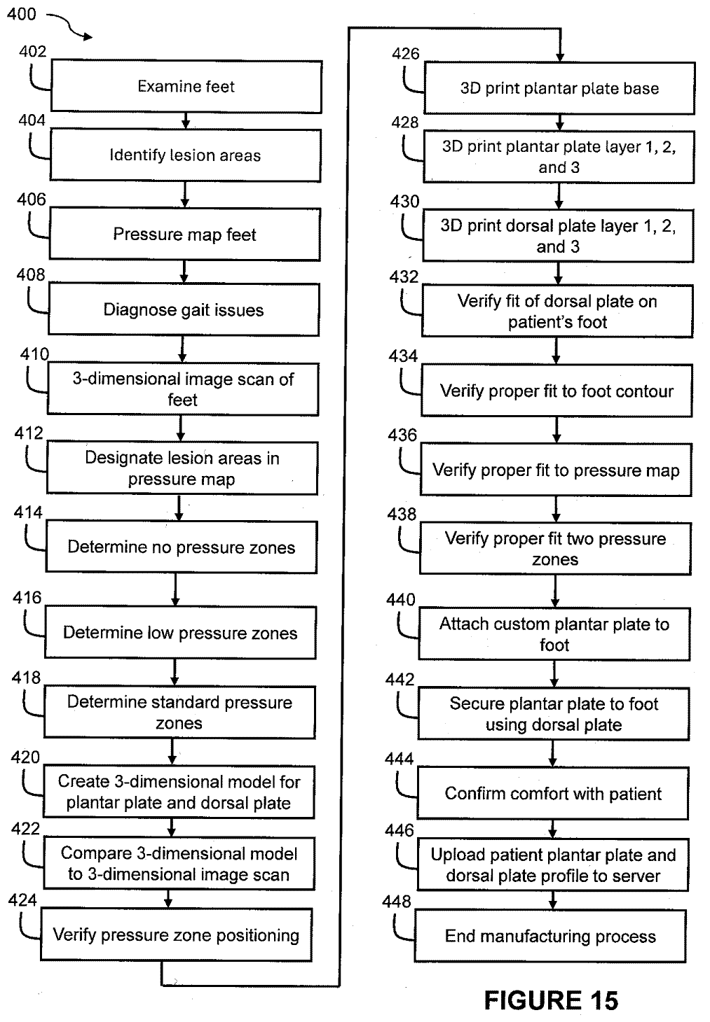

Referring now to FIG. 15, a flow chart showing the manufacturing process for diabetic healing shoe 141 is shown and generally designated as method 400. Method 400 has many of the similar steps as method 200, with one of the main differences being that method 400 includes the manufacturing of dorsal plate 143.

Method 400 starts with step 402 where the physician examines the feet of the patient to determine whether diabetic healing shoe 141 is a viable treatment option for a diabetic foot ulcer. If it is, then at step 404 the different lesion areas are visually identified, and then the pressure map of the feet is created at step 406 and then any gait issues are subsequently diagnosed at step 408.

At step 410 the treating physician can then start the design process of both plantar plate 142 and dorsal plate 143 by having a 3-dimensional image scan of the patient's feet done. Once the scan is complete, then at step 412 the physician designates the different lesion areas on the pressure map, and subsequently identifies all of the different pressure zones that are needed at steps 414, 416, and 418. With all of the different lesion areas and pressure zones identified, then the 3-dimensional model for plantar plate 142 and dorsal plate 143 can be created by the treating physician at step 420. To ensure that the models created are accurate, the treating physician compares the 3-dimensional model to the 3-dimensional image scan at step 422 and then subsequently verifies the pressure zone positioning on plantar plate 142 at step 424.

Once the different design processes are done, then the individual components can begin to be printed at the 3-dimensional print station that is discussed in detail for FIG. 1. First, plantar plate 142 is printed at step 426 and any subsequent layers that are required for printing are done so immediately at step 428. Next, dorsal plate 143 is printed at step 430 and any given number of layers can also be printed in this step.

Step 432 starts the different quality control measures that are in place for method 400 by requiring that the treating physician first verify the fit of dorsal plate 143 on patient's foot. The treating physician then moves to steps 434, 436, and 438 by verifying that plantar plate has the proper foot contour, matches the pressure map, and verifies proper fit.

The final stages of the manufacturing process are then carried out by attaching the plantar plate 142 to the patient's foot at step 440, and then immediately secures dorsal plate 143 to plantar plate 142 at step 442. With both components in place, the treating physician can then verify that the fit is comfortable for the patient at step 444 and make any adjustments that may be needed. Finally, at step 446 both the plantar plate profile and dorsal plate profile are uploaded to the server for reference at a later date before completing the entire manufacturing process at step 448.

It is to be appreciated that the manufacturer of custom plantar plate 142 can be repeated over and over again as the patient's diabetic foot ulcer heals. Specifically, as the ulcer decreases in size the custom plantar plate can be redesigned based upon the current pressure profile that's needed for ongoing healing. Also, as described above, once the healing is complete this same process can be utilized for creating a plantar plate 142 that is used to prevent future ulcers from forming.

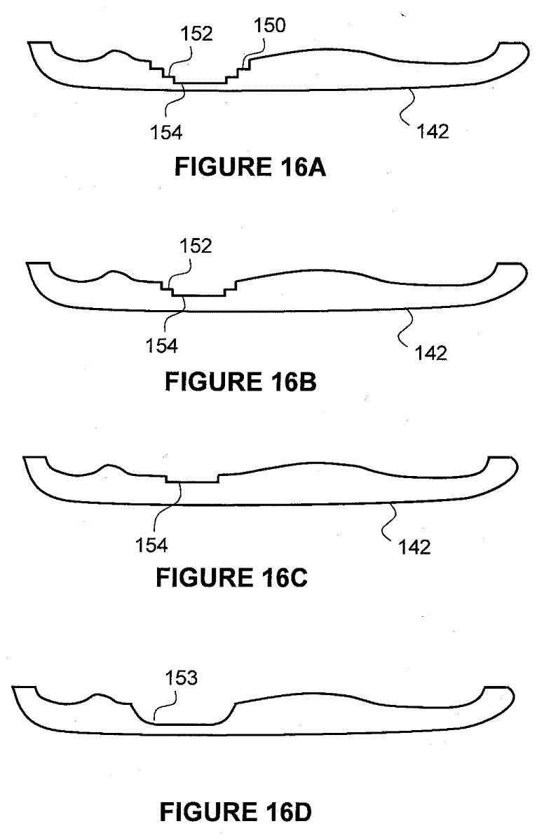

Referring now to FIGS. 16A through 16D, different section views of customer plantar plate 142 are shown. FIG. 16A shows custom plantar plate 142 with three different recessed areas 150, 152, and 154. As a diabetic foot ulcer heals over time, the different offloading requirements may change and only a handful of recessed areas, or one recessed area will be needed as shown in FIG. 16B and FIG. 16C respectively. Recessed areas 150, 152, and 154 are not intended to be limited by the shape shown in FIGS. 16A through 16C as it is fully envisioned that the different areas can have rounded edges, or just have one recess 153 as shown in FIG. 16D. Nor is the number of different recessed areas intended to be limiting. Any given number of different recessed areas can be accommodated into the design of plantar plate 142. This ensures that even the most severe diabetic foot ulcers can be treated through the use of the present invention.

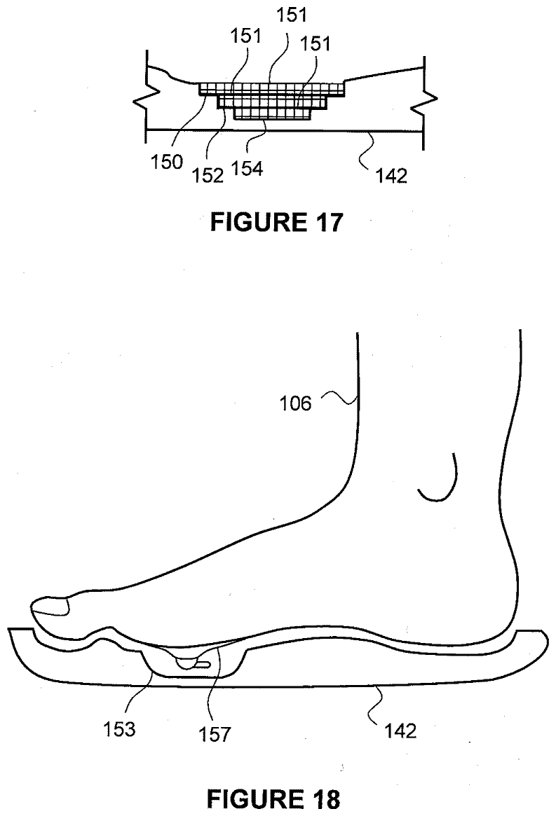

Referring now to FIGS. 17 and 18, different section views of custom plantar plate 142 are shown. Both of the views show different medicants that can be used in conjunction with custom plantar plate 142, but these two figures are not intended to be limiting as custom plantar plate 142 can be customized to accommodate a wide variety of different alternative treatment solutions. FIG. 17 shows custom plantar plate 142 with a number of different gauze packs 151 that can be either medicated or unmedicated to facilitate the healing of a diabetic foot ulcer. FIG. 18 shows custom plantar plate 142 designed to accommodate negative pressure wound therapy device 157 by having one recess 153 in lieu of the different recessed areas.

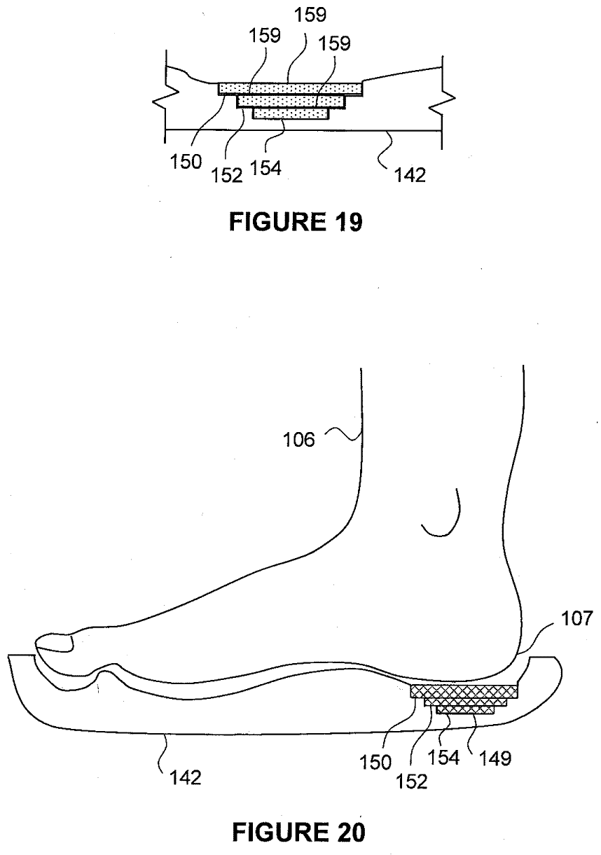

Referring now to FIG. 19, a close-up section view of custom plantar plate 142 having support discs 159 installed within different recessed areas 150, 152, and 154. Support discs 159 may be provided in instances when swelling of foot 106 is a concern for the treating physician. If swelling does occur, then support discs 159 provides enough resistance to foot 106 to prevent the foot from swelling into the different recessed areas. Support discs 159 work in conjunction with the offloading principles of custom plantar plate 142 so that the diabetic foot ulcer present can properly heal.

Referring now to FIG. 20, a section view of custom plantar plate 142 is shown with shock absorbers 149 installed in recessed areas 150, 152, and 154. Shock absorbers 149 may be used in instances where the diabetic foot ulcer is located in a high-pressure area of foot 106; such as heal portion 107. Custom plantar plate 142 is able to offload a majority of the plantar pressures created by foot 106 during normal activity, and shock absorbers 149 absorb the remaining plantar pressure to avoid any irritation to the diabetic foot ulcer. Shock absorbers 149 can be created out of a wide variety of different materials that include EVA foam, polyurethane foam, rubber, or any other material that is currently known in the art.

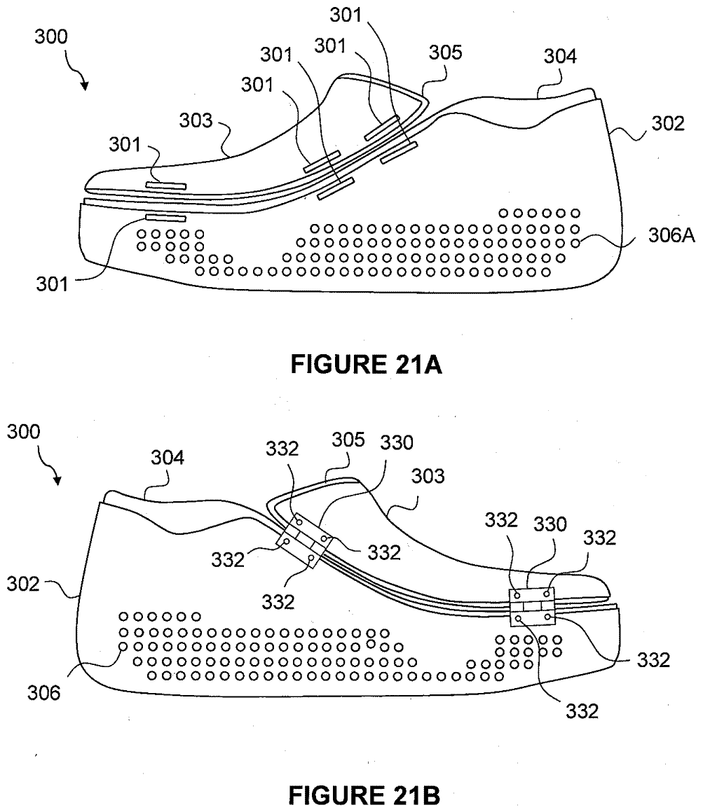

Referring now to FIGS. 21A and 21B, a side views of an alternative embodiment of the diabetic healing shoe is shown fully assembled and generally shown as shoe 300. This alternative embodiment has a number of different components; outer shell 302, dorsum shell 303, lattice shell 304, and dorsum lattice 305.

Outer shell 302 is a protective, load-bearing shell that is custom-fitted to the patient's foot and ankle anatomy that protects the plantar portion of a patient's foot. The upper portion is dorsum shell 303 that operates similar to a clam shell and secures the foot from above so that the foot and ankle are in total alignment. Outer shell 302 is designed to support weight-bearing forces and distribute pressure evenly in a rocker boot. Both outer shell 302 and dorsum shell 303 are custom fit to cover the entire foot and to ensure that the ankle joint can still dorsiflex and plantarflex. Outer shell 302 also has a number of different vent holes 306A along the bottom of the shoe. These vent holes ensure that there is adequate airflow near the wound site to facilitate proper healing.

Lattice shell 304 and dorsum lattice 305 are nestled within outer shell 302 and dorsum shell 303. Both lattice shell 304 and dorsum lattice 305 conforms precisely to the foot's surface, delivering stability and correction at all points except the wound site, which is fully suspended in a zero-contact zone. Further, both lattice shell 304 and dorsum lattice 305 can be easily removed from outer shell 302 and dorsum shell 303 so that updated lattice shells and updated dorsum lattices can be used as healing of the DFU progresses.

FIG. 21A show that on one side both dorsum shell 303 and outer shell 302 have corresponding attachment points 301 to secure the two different pieces together. Attachment points 301 in this view are sized for Velcro straps to slide through, but this is not intended to be limiting as any other fastening mechanism known in the art is fully envisioned. Other fastening mechanisms that are suitable substitutes, include but is not limited to, shoe laces, clap fasteners, magnets and zippers. FIG. 21B shows that the opposite side of both dorsum shell 303 and outershell 302 are connected by hinge 330. The use of hinge 330 ensures that dorsum shell can be easily opened and the patient does not need to worry about placing dorsum shell 303 anywhere. Hinge 330 is attached to outer shell 302 and dorsum shell 303 through the use of multiple screws 332, or other suitable fasteners.

This alternative embodiment has a number of functional features that ensure it can be used daily by a patient. This alternative embodiment provides for gait and stability correct by accounting for vertical limb length discrepancies and correcting gait instability through precise angular and elevation adjustments derived from the initial gait analysis. This includes addressing instabilities in both the ankle and subtalar joints, enabling controlled supination and pronation during motion. The ankle joint of the patient remains free to dorsiflex and plantarflex, allowing natural stair navigation and ambulation without restriction. The device is lightweight and designed for full-time wear, including during sleep and normal indoor activity. The lattice structure allows for water permeability, meaning patients can safely shower with the device on. For additional water protection, a zip-up plastic cover may be used. The device is also easily removable for cleaning, wound inspection, dressing changes, or bathing.

The modular design of the alternative embodiment was done in part to improve patient compliance. The custom device is compatible with a detachable, generic rocker bottom plate that snaps onto the exterior shell. This rocker plate, coated with a high-friction rubberized base for slip resistance, enhances mobility and safety for outdoor use or while driving. Attachment mimics ski boot bindings: the user inserts the toe of the plantar plate into the front of the bottom plate and then secures the heel with a downward push until it locks into place. For removal, the user can step on the back edge of one foot's rocker plate using the other foot to disengage the clip mechanism. This user-friendly, protective footwear system promotes adherence to offloading protocols, reduces treatment duration, and significantly improves clinical outcomes for patients at risk of diabetic foot ulcers.

The device is compatible with embedded sensors, such as those shown and discussed in detail for FIG. 14, that may monitor: plantar pressure and load distribution, skin temperature (for early inflammation detection), wear time (compliance), moisture and microclimate conditions. These sensors can transmit data to clinicians for remote monitoring and to alert patients or providers of any conditions predictive of recurrence. In an advanced embodiment, the plantar platform incorporates a sensor-lined liner capable of detecting pressure abnormalities, temperature fluctuations, and other indicators of potential wound recurrence or pre-ulcerative conditions. These sensors are linked via telemetry, enabling real-time data transmission to the patient's healthcare provider or data retrieval during regular clinic visits. This continuous monitoring enhances early intervention and long-term management of diabetic foot health.

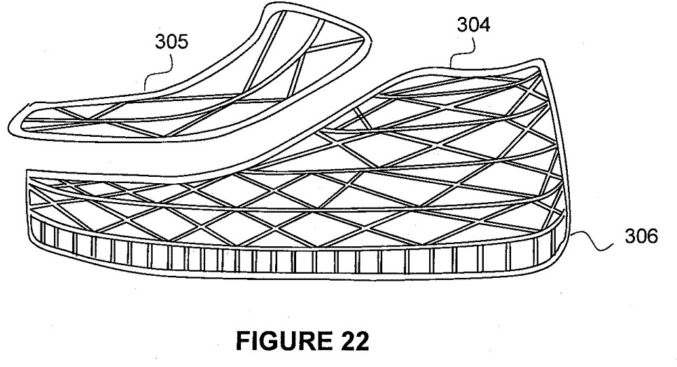

Referring now to FIG. 22, a side view of lattice shell 304 and dorsum lattice 305 are shown. Both lattice shell 304 and dorsum lattice 305 are manufactured once the appropriate scans of a patient's foot are completed. Lattice sole 306 is located along the bottom of lattice shell 304. As can be appreciated from this view, lattice shell 304 and dorsum lattice 305 is a permeable matrix that allows for adequate airflow to ensure that a patient's foot remains dry. Additionally, the stability provided by the lattice structures allow a patient to intermittently remove dorsum shell 303 and outer shell 302 for activities like or similar to showering. Once the patient's foot and lattice structure has fully dried, then dorsum shell 303 and outer shell 302 can be reattached.

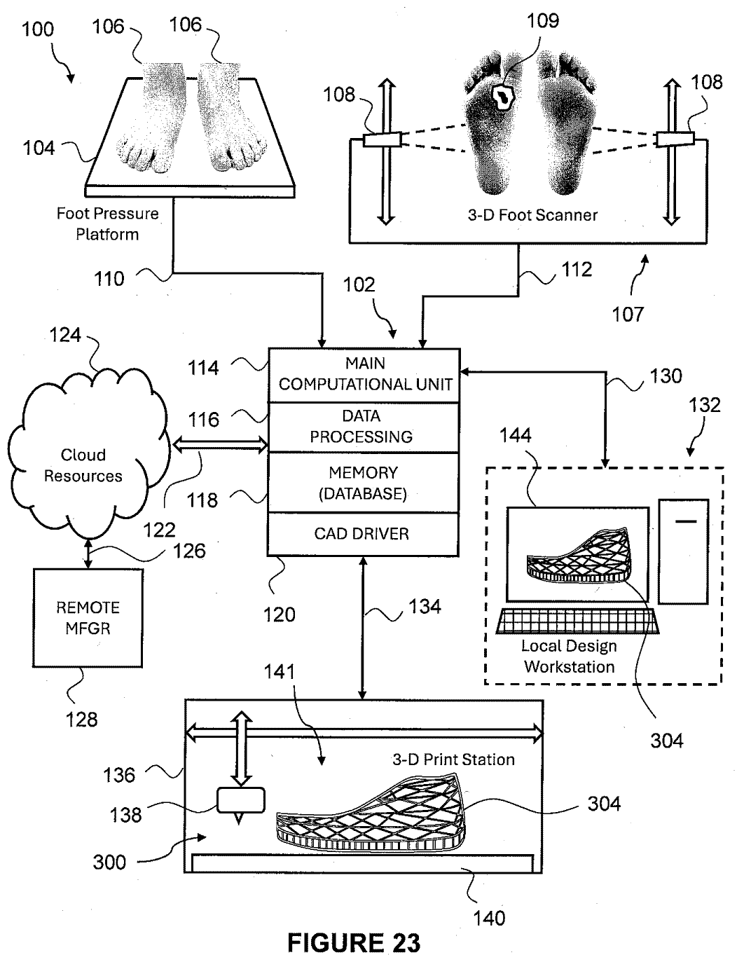

Referring now to FIG. 23, system 100 is shown again, but instead it is shown manufacturing an alternative embodiment of the present invention, diabetic healing shoe 300. Much of the system level discussion that was discussed in detail for FIG. 1 applies again here as system 100 is merely adapted to include the scanning, designing, and manufacturing of dorsal plate outer shell 302, dorsum shell 303, lattice shell 304, dorsum lattice 305, and healing plug 321 (shown in FIG. 31).

FIG. 23 only shows lattice shell 304 on platform 140 and local work station 132, but that is not intended to be limiting. Each piece that makes up diabetic healing shoe 300 needs to be individually manufactured by 3D print station 136 before diabetic healing shoe 300 is assembled. The pieces to be manufactured include dorsal plate outer shell 302, dorsum shell 303, dorsum lattice 305, and healing plug 321 (shown in FIG. 31).

Additionally, the location of diabetic foot ulcer 109 is not intended to be limiting. It is fully envisioned that shoe 300 can be used to treat diabetic foot ulcers that are located on any surface of foot 106. This includes, but is not limited to, the plantar, dorsal, medial, lateral, posterior sides, or even the toes of foot 106.

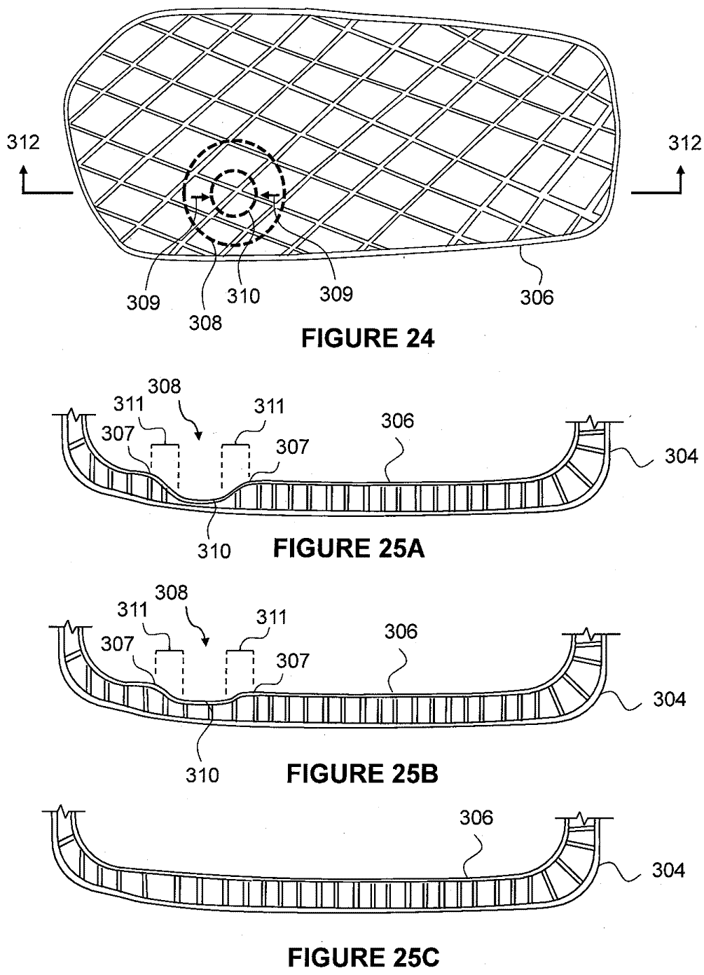

FIGS. 24-28 show different possible locations for recessed area 308. Recessed area 308 can be located anywhere within the interior surface of lattice shell 304 or the interior surface of dorsum lattice 305. Both lattice shell 304 and dorsal lattice 305 are custom printed based on the needs of a specific patient, so recessed area 308 can be located and sized to the specific wound. The different locations of recessed area 308 are on the plantar portion of lattice shell 304, a side wall of lattice shell 304, and on dorsum lattice 305. However, these different locations are not intended to be limiting as recessed area 308 can be located anywhere on a patient's foot, including the plantar, dorsal, medial, lateral, or posterior sides. Additionally, recessed area 308 can be located to treat diabetic foot ulcers on a patients toes and recessed area 308 can be partially located on lattice shell 304 and dorsum lattice 305.

Referring now to FIGS. 24 and 25A through 25C, a top view and multiple section views of lattice sole 306 are shown. These different views provide a general overview of how a treatment process may progress overtime. Lattice sole 306 is shown in a matrix configuration with recessed area 308 fully incorporated into lattice sole 306. Recessed area 308 follows a gradual downward slope along direction 309 until it levels out at center 310. Slope gradient 311 can be adjusted based on the specific requirements of an individual patient, but outer boundary 307 (shown in FIGS. 25A through 25C) that demarcates the beginning of slope gradient 311 almost always forms a rounded edge to ensure that plantar pressures are properly offloaded. Consequently, recessed area 308 and center 310 can either have a wider or narrower diameter depending on the size and severity of an individual foot ulcer. It is also fully envisioned that recessed area 308 can take on any shape necessary to treat a diabetic foot ulcer and recessed area 308 is not intended to be limited by the circular shape shown.

FIGS. 25A through 25C show section views along line 312 in FIG. 24 to show the different healing stages for a diabetic foot ulcer. FIG. 25A shows slope gradient 311 at its steepest grade during the initial phases of treatment. As the diabetic foot ulcer heals over time, then slope gradient 311 can begin to level out as shown in FIG. 25B. Recessed area 308 may no longer be needed once the healing of the diabetic foot ulcer is complete as shown in FIG. 25C. Lattice sole 306 as shown in FIG. 25C may also be used for preventative treatment of diabetic foot ulcer. If a supervising physician decides that a patient is a high risk for a foot ulcer, then present invention can still be prescribed and used for preventative care.

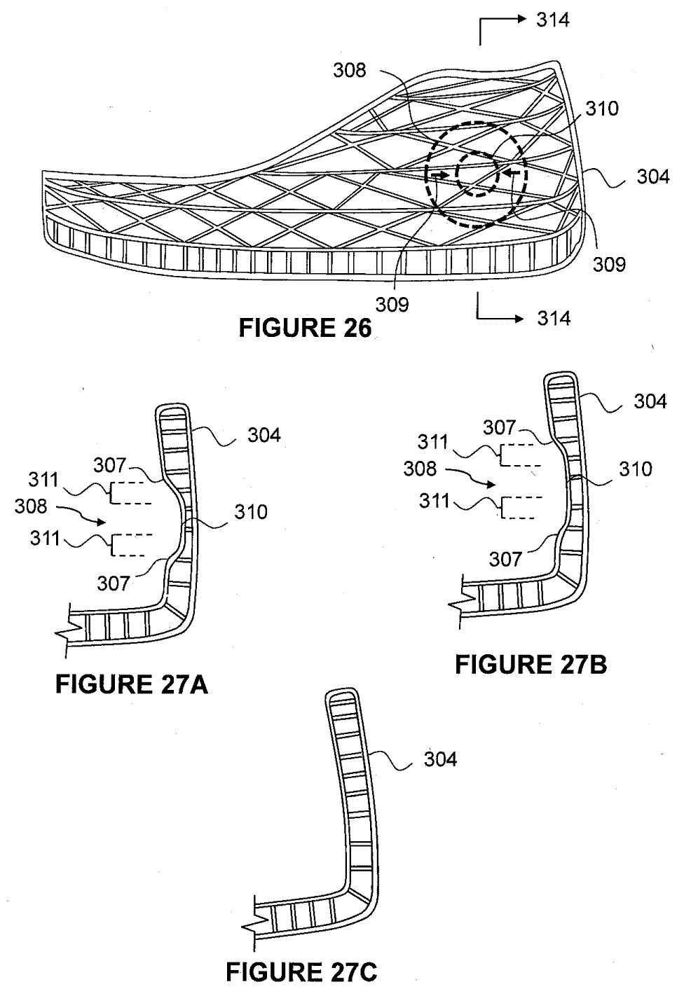

Referring now to FIGS. 26 and 27A through 27C show recessed area 308 printed onto a side of lattice shell 304. The following disclosures apply equally regardless of what side of lattice shell 304 that recessed area 308 is located. It is fully envisioned that recessed area can be located on either the anterior side, medial side, lateral side, or posterior side of lattice shell 304.

Recessed area 308 follows a gradual downward slope along direction 309 until it levels out at center 310. Slope gradient 311 can be adjusted based on the specific requirements of an individual patient, but outer boundary 307 (shown in FIGS. 27A through 27C) that demarcates the beginning of slope gradient 311 almost always forms a rounded edge to ensure that plantar pressures are properly offloaded. Consequently, recessed area 308 and center 310 can either have a wider or narrower diameter depending on the size and severity of an individual foot ulcer. It is also fully envisioned that recessed area 308 can take on any shape necessary to treat a diabetic foot ulcer and recessed area 308 is not intended to be limited by the shape shown.

FIGS. 27A through 27C are section views along line 314 in FIG. 26 that show recessed area 308 at different stages of the healing process. FIG. 27A shows slope gradient 311 at its steepest grade during the initial phases of treatment. As the diabetic foot ulcer heals over time, then slope gradient 311 can begin to level out as shown in FIG. 27B. Recessed area 308 may no longer be needed once the healing of the diabetic foot ulcer is complete as shown in FIG. 27C. Lattice shell 304 as shown in FIG. 27C may also be used for preventative treatment of diabetic foot ulcer. If a supervising physician decides that a patient is a high risk for a foot ulcer, then present invention can still be prescribed and used for preventative care.

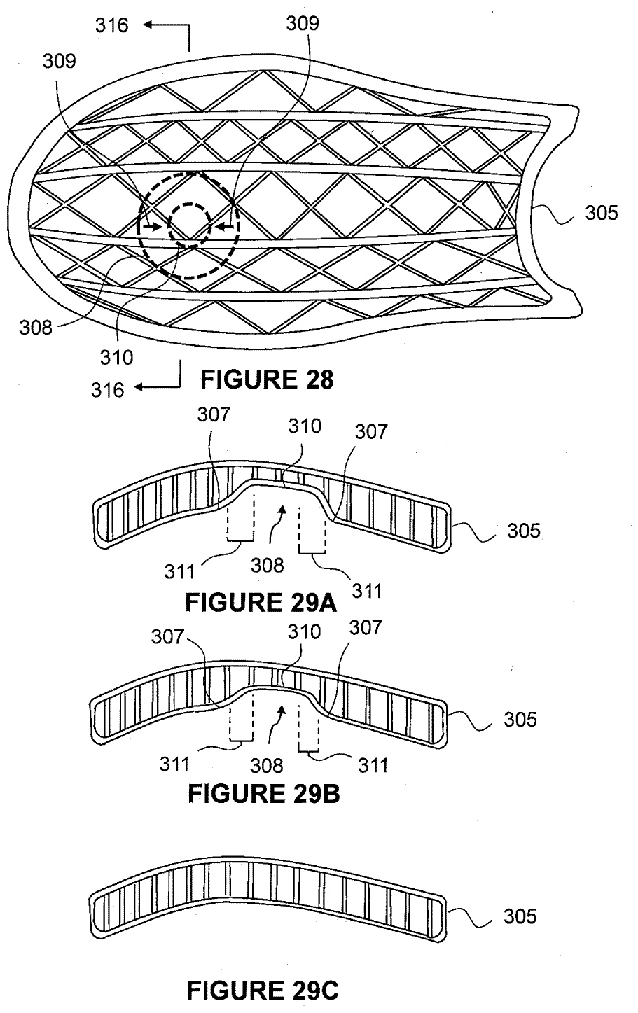

Referring now to FIGS. 28 and 29A through 29C, show recessed area 308 printed into the interior surface of dorsum lattice 305. Recessed area 308 can be located anywhere within the interior surface of dorsum lattice 305. It is also fully envisioned that recessed area 310 can be partially located on the interior surface of dorsum lattice 305 and the interior surface of lattice shell 304 if needed. Recessed area 308 follows a gradual downward slope along direction 309 until it levels out at center 310. Slope gradient 311 can be adjusted based on the specific requirements of an individual patient, but outer boundary 307 (shown in FIGS. 29A through 29C) that demarcates the beginning of slope gradient 311 almost always forms a rounded edge to ensure that plantar pressures are properly offloaded. Consequently, recessed area 308 and center 310 can either have a wider or narrower diameter depending on the size and severity of an individual foot ulcer. It is also fully envisioned that recessed area 308 can take on any shape necessary to treat a diabetic foot ulcer and recessed area 308 is not intended to be limited by the shape shown.

FIGS. 29A through 29C are section views along line 316 in FIG. 28 that show recessed area 308 at different stages of the healing process. FIG. 29A shows slope gradient 311 at its steepest grade during the initial phases of treatment. As the diabetic foot ulcer heals over time, then slope gradient 311 can begin to level out as shown in FIG. 29B. Recessed area 308 may no longer be needed once the healing of the diabetic foot ulcer is complete as shown in FIG. 29C. Lattice shell 304 as shown in FIG. 29C may also be used for preventative treatment of a diabetic foot ulcer. If a supervising physician decides that a patient is a high risk for a foot ulcer, the present invention can still be prescribed and used for preventative care.

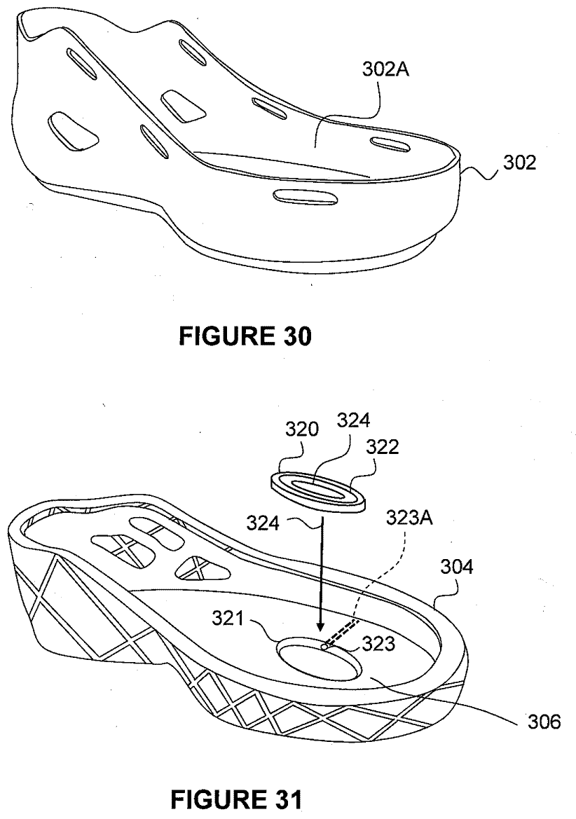

Referring now to FIGS. 30 and 31, perspective views of outer shell 302 and lattice shell 304 are shown. In these views, both components are shown separated from each other. To reassemble the two components, lattice shell 304 needs to be placed back within the interior 302A of outer shell 302 to form a snug fit.

Specific to FIG. 31, lattice shell 304 is shown with healing plug 320. Healing plug 320 fits within healing area 321 formed in lattice sole 306, and has different recessed areas 322 and 324. Healing plug 320 is placed within healing area 321 by pushing down on healing plug 320 along direction 324. Healing area 321 has a slightly wider diameter than healing plug 320 to ensure that healing plug 320 snugly fits within healing area 321 and cannot fall out during the ordinary course of use. Healing plug 320 is a custom plug that is fabricated to the exact size and shape of the diabetic foot ulcer that is being treated, so the circular shape shown here is not intended to be limiting. When healing plug 320 is used, it is the only component that needs to be reprinted as the diabetic foot ulcer heals over time. Healing plug 320 offloads all of the pressure experienced by the diabetic foot ulcer and mimics a zero contact environment through recessed areas 322 and 324.

Healing plug 320 may also be used as an open cavity, of exact shape and size of diabetic foot ulcer 109, to house any type of wound dressings, biologics (skin substitute), wound VAC foam that comes into direct contact with the wound base. Additionally, the lattice shell can be printed with channels to house tubing used for VAC (negative pressure) system. When no longer needed, the tube channels will be filled back up with inner lattice material.

Healing area 321 in this view is located in the lattice sole 306 of lattice shell 304 and is an aperture that is designed for healing plug 320 to fit inside of. A user just needs to apply pressure either from the top or bottom of healing pad 320 in order to fully remove it from healing area 321. In this embodiment only healing pad 320 needs to be reprinted to adjust the different recessed areas 322 and 324 as the healing process of a diabetic foot ulcer progresses. There are also embodiments that fully envision recessed area 324 as an aperture to accommodate different types of medicants, biologics, topicals and even foam used in negative pressure wound therapy devices. The tubing for a negative pressure wound therapy device can run through hole 323 and channel 323A for connection to a vacuum source. When the VAC (negative pressure) is no longer needed, the tubing may be removed, and the channels for the tubing can be filled in with lattice. Healing plug 320 and healing area 321 are shown here on lattice sole 306, but it is fully envisioned that healing plug 320 and healing area 321 and all of the accompanying disclosures can be adapted for a side of lattice shell 304 and dorsum lattice 305 as shown and described in FIGS. 26-29C.

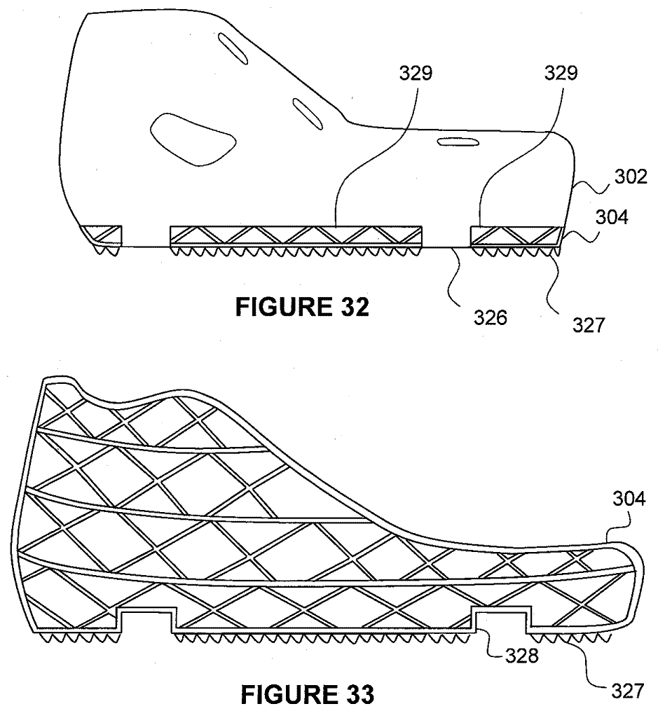

Referring now to FIGS. 32 and 33, an alternative embodiment of outer shell 302 and lattice shell 304 are shown. In this alternative embodiment, lattice shell 304 will protrude or come through designated openings 329 in the plantar surface of outer shell 302 to provide additional traction. The additional traction is provided by treading 327 which is printed as a fully integrated component of lattice shell 304. Each lattice shell 304 is printed with recesses 328 to accommodate extrusion 326 on plantar plate 302. In this configuration, the outer shell provides an external structure to lattice shell 304, yet provides improved traction due to the exposure of treading 327.

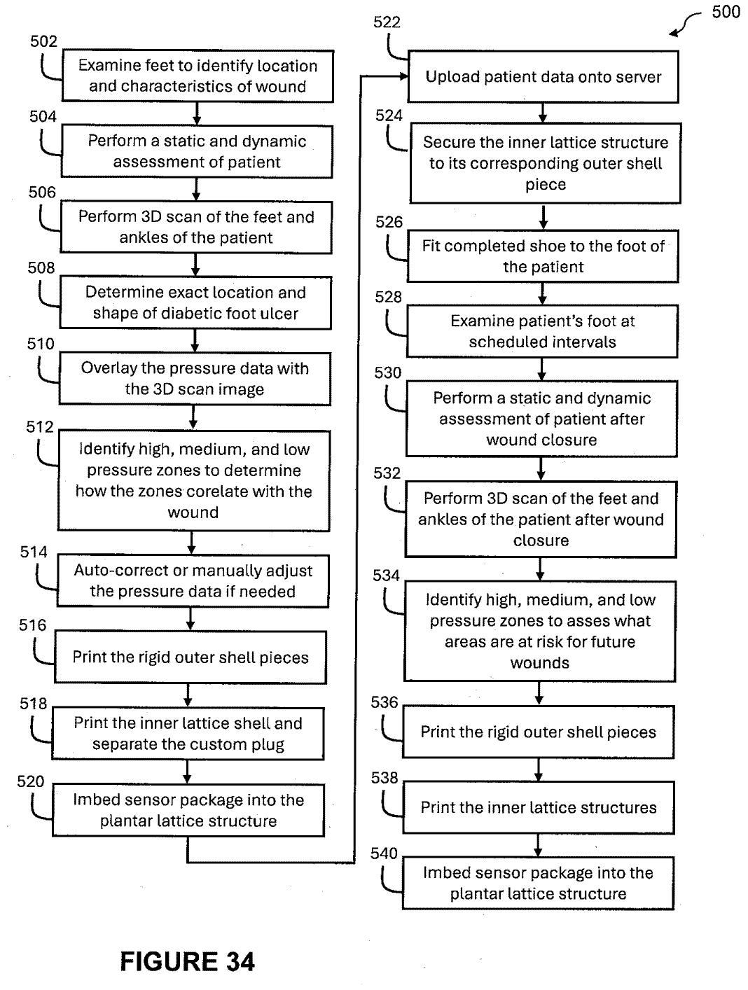

Referring now to FIG. 34, a flow chart that outlines an exemplary order of operations for shoe 300 and generally designated as method 500 is shown. This order of operations is not intended to be limiting as any of the given steps can performed in any order, or simultaneously if need be.

Starting with step 502, a healthcare provider begins by performing a full foot and ankle exam to determine the shape of a patient's foot, determine the location of any deformities, make note of any swelling areas, and identify the location and characteristics of the diabetic foot ulcer. The patient then steps onto the pressure plate at step 504 so that the healthcare provider can perform static and dynamic assessments of the patient's foot. Some of the features that are evaluated during step 504 are the balance or stability of the patient, the patient's center of gravity, and recordation of any limb length and/or gait discrepancies.

At step 506, a full 3D scan of the patient's feet and ankles are done. This includes taking separate scans of the feet and ankles when they are fully weightbearing, not weightbearing, or when they are only partially weightbearing. This ensures that the healthcare provider can develop a clear picture of how the foot shape changes during weightbearing, identify any pressure areas, and to accurately determine the exact location and dimensions of the wound. Step 508 is then done to determine the exact location, size and shape of the wound.

At step 510, the healthcare provider can then overlay the pressure data collected onto the 3D scan image by superimposing both images. This ensures that the healthcare provider can see how the pressure map directly relates with the location of the diabetic foot ulcer. The healthcare provider then moves to step 512 to identify the high, medium, and low pressure zones and determine how each of those zones correlate with the location of the diabetic foot ulcer. If there are any abnormal pressure readings, the healthcare provider is able to manually or use the autocorrect feature to make any adjustments to the pressure map at step 514 to achieve a perfect fit. Steps 510 to 514 provide one example of how each diabetic healing shoe is customized to the unique needs of an individual patient.

The different pieces of the diabetic healing shoe can be printed once the healthcare provider is satisfied with the overlay of the pressure map and the 3D image scan. There are two different shells, the rigid outer shell (outer shell 302 and dorsum shell 303) and the inner lattice structure (lattice shell 304, dorsum lattice 305, and healing plug 320), that need to be printed after all of the data and manual adjustments are made. Further, all of these individual pieces, including healing plug 320, are printed individually. These different pieces cannot be printed earlier on in the process because each piece that is printed is fully customized to the affected foot and individualized for every patient. The outer shell is rigid and used for ground contact and housing the lattice shell. The inner lattice shell is made up of various shore densities that provide total contact of the entire foot/ankle and toes, correction for pressure points wherever needed, zero contact full offloading of foot ulcer, and total protection of every aspect of a patient's foot.

The printing process for the outer shell pieces are covered by step 516, and the printing of the lattice shell pieces, including the healing plug, is covered by step 518. The printing of the lattice shell is critical due to the different features of the shell. The lattice shell will correct the pressure abnormalities noted from data collected during pressure mapping and the gait and balance assessment, and it can have a follow hollow cavity near the vicinity of the open wound found on any part of the foot, ankle, and/or toes. When installed, healing plug 321 does not have any direct contact with the wound to ensure that proper offloading at the wound site occurs. When used, healing plug 320 can be easily removed by a health care practitioner and reprinted as the healing process of the diabetic foot ulcer progresses.

At step 520, the healthcare provider can imbed a sensor package directly into the lattice shell. The types of sensors that could be used include, but are not limited to, temperature sensor, pressure sensors, and patient compliance sensors.

With all of the different components completed, the healthcare provider can then upload all of the relevant patient data onto a server at step 522 for future use, reprint or modifications. The printed components of the device will then be assembled together at step 524. The lattice shell is secured to the interior facing side of the corresponding outer shell piece by using an adhesive or glue, or rely only on a friction fit, to firmly secure the lattice shell in place. The healthcare provider can then proceed to step 526 and fit the shoe directly onto the affected foot of the patient. The patient is scheduled for subsequent examinations at different intervals at step 528 so that the healthcare provider can ensure that the diabetic foot ulcer is healing properly.

Once the diabetic foot ulcer is fully healed, then the preventative stage outlined in steps 530 to 540 can be followed to have a preventative diabetic foot ulcer shoe made. At step 530, the healthcare provider performs another static and dynamic assessment of the patient and then performs another 3D scan of the patient's feet and ankles at step 532. The data collected at steps 530 and 532 allow a healthcare provider to determine any high risk areas of a patient's foot that are susceptible to develop another or new diabetic foot ulcers. For example, at step 534 the healthcare provider can once again determine the different high, medium, and low pressure zones. High pressures at certain areas of a foot may lead to higher risk of recurrence and potentially developing diabetic foot ulcers in the future. The different components of the preventative diabetic foot ulcer shoe can then be printed, with the outer shell printed at step 536 and the lattice shell printed at step 538. The lattice shell 538 can also have different sensors imbedded into it in step 540 in a similar manner to the sensors used at step 520.

Various features of preferred embodiments of the present invention have been discussed in connection with certain embodiments for the sake of clarity and ease of understanding. Further embodiments incorporating the possible combinations of features described above in connection with specific embodiments are fully contemplated herein.

While there have been shown what are presently considered to be preferred embodiments of the present invention, it will be apparent to those skilled in the art that various changes and modifications can be made herein without departing from the scope and spirit of the invention.

I claim:

1. A shoe comprising:
an outer shell to encase a foot of an individual;
an outer dorsal shell to encase a top of the foot of the patient;
an inner lattice shell configured to fit within the outer shell;
an inner dorsal lattice configured to fit within the outer dorsal shell; and
a fastening mechanism that secures the outer shell and the outer dorsal to the foot of the individual.

2. The shoe of claim 1, wherein the inner lattice shell further comprises at least one recessed area.

3. The shoe of claim 2, wherein the at least one recessed area is located on a sole of the inner lattice shell.

4. The shoe of claim 2, wherein the at least one recessed area is located on a side of the inner lattice shell.

5. The shoe of claim 1, wherein the inner lattice shell further comprises at least one healing area defined by an aperture and at least one healing plug that is sized to fit within the at least one healing area.

6. The shoe of claim 5, wherein the at least one healing area and at least one healing plug is located on a sole of the inner lattice shell.

7. The shoe of claim 5, wherein the at least one healing area and at least one healing plug is located on a side of the inner lattice shell.

8. The shoe of claim 1, wherein the inner dorsal lattice further comprises at least one recessed area.

9. The shoe of claim 1, wherein the inner dorsal lattice further comprises at least one healing area that is defined by an aperture and at least one healing plug that is sized to fit within the at least one healing area.

10. A shoe comprising:
an outer shell and an inner lattice shell secured to an interior side of the outer shell such that the outer shell and the inner lattice shell encase a foot of an individual;
an outer dorsal shell and an inner dorsal lattice secured to an interior side of the outer dorsal shell such that the outer dorsal shell and the inner dorsal lattice encase the top of the foot of the individual; and
a fastening mechanism that secures the outer shell and the outer dorsal to the foot of the individual.

11. The shoe of claim 10, wherein the inner lattice shell further comprises at least one recessed area.

12. The shoe of claim 11, wherein the at least one recessed area is located on a sole of the inner lattice shell.

13. The shoe of claim 11, wherein the at least one recessed area is located on a side of the inner lattice shell.

14. The shoe of claim 10, wherein the inner lattice shell further comprises at least one healing area defined by an aperture and at least one healing plug that is sized to fit within the at least one healing area.

15. The shoe of claim 14, wherein the at least one healing area and at least one healing plug is located on a sole of the inner lattice shell.

16. The shoe of claim 14, wherein the at least one healing area and at least one healing plug is located on a side of the inner lattice shell.

17. The shoe of claim 10, wherein the inner dorsal lattice further comprises at least one recessed area.

18. The shoe of claim 10, wherein the inner dorsal lattice further comprises at least one healing area that is defined by an aperture and at least one healing plug that is sized to fit within the at least one healing area.
